(19) United States
(12) Patent Application Publication
(10) Pub. No.: US 2015/0313947 A1
(43) Pub. Date: Nov. 5, 2015

(54) TREATMENT OF SCHIZOPHRENIA USING AMNION DERIVED ADHERENT CELLS

(71) Applicant: ANTHROGENESIS CORPORATION, Warren, NJ (US)

(72) Inventors: Robert J. Hariri, Bernardsville, NJ (US); Jodi P. Gurney, Chicago, IL (US)

(21) Appl. No.: 14/647,491

(22) PCT Filed: Dec. 2, 2013
(86) PCT No.: PCT/US2013/072562

§ 371 Date: May 27, 2015

Related U.S. Application Data
(60) Provisional application No. 61/732,849, filed on Dec. 3, 2012.

(51) Int. Cl. A61K 35/50

(52) U.S. Cl. CPC A61K 35/50

(57) ABSTRACT

Provided herein are methods of treatment of individuals having schizophrenia, or schizophreniform disorder, using angiogenic cells from amnion, referred to as amnion derived adherent cells, and populations of and compositions comprising, such cells. A need exists for therapies that can ameliorate or palliate schizophrenia, or a schizophreniform disorder, or symptoms of the same. Provided herein are methods of treating individuals who have schizophrenia, or schizophreniform disorder, comprising administration of therapeutically-effective amounts (numbers) of AMDACs.

This application claims priority to U.S. Provisional Patent Application No. 61/732,849, filed Dec. 3, 2012, the disclosure of which is herein incorporated by reference in its entirety.

### 1. FIELD

Provided herein are methods of treating individuals having schizophrenia, or a schizophreniform disorder, comprising administering to the individual a therapeutically effective amount of angiogenic cells from amnion, referred to herein as “amnion derived adherent cells” (AMDACs). Amnion derived adherent cells are distinct from previously-described tissue culture surface-adherent placental stem cells.

### 2. BACKGROUND

A need exists for therapies that can ameliorate or palliate schizophrenia, or a schizophreniform disorder, or symptoms of the same. Provided herein are methods of treating individuals who have schizophrenia, or schizophreniform disorder, comprising administration of therapeutically-effective amounts (numbers) of AMDACs.

### 3. SUMMARY

In one aspect, provided herein is a method of treating schizophrenia comprising administering to an individual having schizophrenia a therapeutically effective amount of amnion-derived adherent cells (AMDACs), or medium conditioned by AMDACs. In certain embodiments, “therapeutically effective” means an amount effective to reduce or ameliorate one or more symptoms of schizophrenia that manifest in the individual. In certain embodiments, said one or more symptoms comprise one or more Schneiderian First Rank symptoms, e.g., hearing thoughts audibly; hearing voices arguing; hearing voices commenting on one's actions; delusions of being controlled by others; delusions that the individual's thoughts have been withdrawn or taken by others; delusions that thoughts have been inserted or caused by others; delusions that others can read the individual's thoughts; and/or delusional perception by the individual. In certain embodiments, said one or more symptoms comprise one or more of disordered thoughts and/or speech; formal thought disorder; hallucinations (e.g., tactile, auditory, visual, olfactory and/or gustatory hallucinations); flat or blunted affect and/or emotion; poverty of speech (alogia); inability to experience pleasure (anhedonia); lack of desire to form relationships (asociality); and/or lack of motivation (avolition). In certain embodiments, the individual is diagnosed as schizophrenic according to either or both of Diagnostic and Statistical Manual of Mental Disorders (e.g., version DSM-IV-TR), or the World Health Organization's International Statistical Classification of Diseases and Related Health Problems (ICD-10) criteria. In certain specific embodiments, said one or more symptoms of formal thought disorder comprise one or more of blocking (interrupted speech, often with no memory of the discussed topic after the block); circumstantiality (highly detailed speech that delays reaching a goal); clanging (speech emphasizing sounds, rather than meaning); derailment (speech characterized by failure to maintain ideas on a single track); distractible speech; echolalia (echoing of speech once, or in continuous in repetition); evasive interaction (expression of ideas and/or feelings about another individual evasively or in diluted form); flight of ideas (speech characterized by a sequence of unrelated ideas); illogicality (non-sequiturs or faulty inferences); incoherence (word salad); loss of goal (failure to show a train of thought to a natural conclusion); neologisms (new word formations); perseveration (repetition of words or ideas); honemic paraphasia; pressured speech (increase in the rate or amount of spontaneous speech); self-reference (speaker's frequent and inappropriate reference back to self); semantic paraphasia; stilted speech; tangentiality (speech characterized by an oblique, tangential or irrelevant manner); and/or word approximations (old words used in a new or unconventional way).

In another aspect, provided herein is a method of prophylaxis against schizophrenia in an infant, comprising prenatally administering AMDACs, e.g., an effective dose of AMDACs, to an individual pregnant with the infant. In certain embodiments, the individual's immune system has been activated during pregnancy by, e.g., bacterial infection, viral infection, rickettsial infection, spirochete infection, protozoal infection, exposure to allergens, or the like. In certain embodiments, the individual who gives birth to the infant has an infection during pregnancy, e.g., a bacterial infection, viral infection, rickettsial infection, spirochete infection, or protozoal infection. In a specific embodiment, said viral infection is a viral infection during the first trimester. In another specific embodiment, said bacterial infection is a bacterial infection in the second trimester.

In another aspect, provided herein is a therapeutically effective amount of AMDACs, or culture medium conditioned by AMDACs, for use in treating schizophrenia in an individual, wherein the therapeutically effective amount is an amount sufficient to cause a detectable improvement in one or more symptoms of schizophrenia manifested by said individual. In certain embodiments, said individual has been diagnosed as having schizophrenia according to criteria listed in the Diagnostic and Statistical Manual of Mental Disorders (DSM). In other specific embodiments, said one or more symptoms comprise one or more Schneiderian First Rank symptoms. In other specific embodiments, said one or more symptoms comprise one or more of hearing thoughts audibly; hearing voices arguing; hearing voices commenting on one's actions; delusions of being controlled by others; delusions that the individual's thoughts have been withdrawn or taken by others; delusions that thoughts have been inserted or caused by others; delusions that others can read the individual's thoughts; or delusional perception by said individual. In certain other embodiments, said one or more symptoms comprise one or more of disordered thoughts and/or speech; tactile hallucinations; auditory hallucinations; visual hallucinations; olfactory hallucinations; gustatory hallucinations); flat or blunted affect; flat or blunted emotion; poverty of speech; inability to experience pleasure; lack of desire to form relationships; or lack of motivation.

In certain embodiments, said AMDACs are HLA-G⁻, as determinable by RT-PCR. In certain other specific embodiments, said AMDACs are additionally CD49f⁺, as determinable by immunolocalization. In certain other specific embodiments, said AMDACs are OCT-4⁻, HLA-G⁻ and CD49f⁺. In other specific embodiments, said AMDACs are CD90⁺, CD105⁺, or CD117⁻ as determinable by immunolocalization. In another specific embodiment, said AMDACs are CD90⁺, CD105⁺, and CD117⁻ as determinable by flow cytometry. In a more specific embodiment, said AMDACs are OCT-4⁻ and HLA-G⁻, as determined by RT-PCR, and CD49f⁺, CD90⁺, CD105⁺, and CD117⁻ as determinable by immunolocalization. In another specific embodiment, said AMDACs are VEGFR1/Flt-1⁺ (vascular endothelial growth factor receptor 1) and VEGFR2/KDR⁺ (vascular endothelial growth factor receptor 2), as determinable by immunolocalization. In another specific embodiment, said AMDACs are one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻ (angiotensin-I-converting enzyme, ACE), CD146⁻ (melanoma cell adhesion molecule), or CXCR4⁻ (chemokine (C-X-C motif) receptor 4) as determinable by immunolocalization. In another specific embodiment, said AMDACs are CD9⁺ CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, and CXCR4⁻ as determinable by immunolocalization. In another specific embodiment of any of the above embodiments, the AMDACs are VE-cadherin⁻ as determinable by immunolocalization. In another specific embodiment, said AMDACs are additionally positive for CD105⁺ and CD200⁺ as determinable by immunolocalization. In another specific embodiment, said AMDACs do not express CD34 as determinable by immunolocalization after exposure to 50 ng/mL VEGF for 7 days.

In certain embodiments, the AMDACs are comprised within an isolated population of cells, wherein at least 50%, 80%, or 90% of the cells in said population are the cells of claim 1.

In other specific embodiments, the AMDACs, useful to treat schizophrenia or schizophreniform disorder, are adherent to a tissue culture surface; wherein said AMDACs are OCT-4⁻, as determinable by RT-PCR, and CD49f⁺, HLA-G⁻, CD90⁺, CD105⁺, and CD117⁻, as determinable by immunolocalization; and wherein said AMDACs: (a) express one or more of CD9, CD10, CD44, CD54, CD98, CD200, Tie-2, TEM-7, VEGFR1/Flt-1, or VEGFR2/KDR (CD309), as determinable by immunolocalization; (b) lack expression of CD31, CD34, CD38, CD45, CD133, CD143, CD144, CD146, CD271, CXCR4, HLA-G, or VE-cadherin, as determinable by immunolocalization, or lack expression of SOX2, as determinable by RT-PCR; (c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR; (d) express one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; (e) secrete VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the AMDACs are cultured; (f) express micro RNAs miR-17-3p, miR-18a, miR-18b, miR-19b, miR-92, or miR-296 at a higher level than an equivalent number of bone marrow-derived mesenchymal stem cells; (g) express micro RNAs miR-20a, miR-20b, miR-221, miR-222, miR-15b, or miR-16 at a lower level than an equivalent number of bone marrow-derived mesenchymal stem cells; (h) express miRNAs miR-17-3p, miR-18a, miR-18b, miR-19b, miR-92, miR-20a, miR-20b, miR-296, miR-221, miR-222, miR-15b, or miR-16; and/or (i) express increased levels of CD202b, IL-8 or VEGF when cultured in less than about 5% O₂, compared to expression of CD202b, IL-8 or VEGF under 21% O₂.

The isolated amnion derived adherent cells and cell populations provided herein are not the isolated placental stem cells or cell populations described, e.g., in U.S. Pat. Nos. 7,468,276 or 7,255,879, or U.S. Patent Application Publication No. 2007/0275362. The isolated amnion derived adherent cells provided herein are also not endothelial progenitor cells, amniotic epithelial cells, trophoblasts, cytotrophoblasts, embryonic germ cells, embryonic stem cells, cells obtained from the inner cell mass of an embryo, or cells obtained from the gonadal ridge of an embryo.

As used herein, the term “about” means, e.g., within 10% of a stated figure or value.

As used herein, the term “derived” means isolated from or otherwise purified. For example, amnion derived adherent cells are isolated from amnion. The term “derived” encompasses cells that are cultured from cells isolated directly from a tissue, e.g., the amnion, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “isolated cell” means a cell that is substantially separated from other, cells of the tissue, e.g., amnion or placenta, from which the cell is derived. A cell is “isolated” if at least about 20%, 30%, 40%, 50%, 60%, 70%, 80%, 90%, 95%, or at least 99% of the cells with which the stem cell is naturally associated are removed from the cell, e.g., during collection and/or culture of the cell.

As used herein, the term “isolated population of cells” means a population of cells that is substantially separated from other cells of the tissue, e.g., amnion or placenta, from which the population of cells is derived. A cell is “isolated” if at least about 20%, 30%, 40%, 50%, 60%, 70%, 80%, 90%, 95%, or at least 99% of the cells with which the population of cells, or cells from which the population of cells is derived, is naturally associated are removed from the cell, e.g., during collection and/or culture of amnion derived adherent cells.

As used herein, a cell is “positive” for a particular marker when that marker is detectable above background, e.g., by immunolocalization, e.g., by flow cytometry; or by RT-PCR. For example, a cell is described as positive for, e.g., CD105 if CD105 is detectable on the cell in an amount detectably greater than background (in comparison to, e.g., an isotype control). In the context of, e.g., antibody-mediated detection, “positive,” as an indication a particular cell surface marker is present, means that the marker is detectable using an antibody, e.g., a fluorescently-labeled antibody, specific for that marker; “positive” also means that a cell bears that marker in a amount that produces a signal, e.g., in a cytometer, that is detectably above background. For example, a cell is “CD105⁺” where the cell is detectably labeled with an antibody specific to CD105, and the signal from the antibody is detectably higher than a control (e.g., background). Conversely, “negative” in the same context means that the cell surface marker is not detectable using an antibody specific for that marker, e.g., as compared to background or to an isotype control. For example, a cell is “CD34⁻” where the cell is not detectably labeled with an antibody specific to CD34 as compared to an isotype control. Unless otherwise noted herein, cluster of differentiation (“CD”) markers are detected using antibodies. For example, OCT-4 can be determined to be present, and a cell is OCT-4⁺, if mRNA for OCT-4 is detectable using RT-PCR, e.g., for 30 cycles.

### 4. DETAILED DESCRIPTION

### 4.1 Treatment of Schizophrenia

In one aspect, provided herein is a method of treating an individual who has schizophrenia, or a schizophreniform disorder, comprising administering to the individual a therapeutically-effective amount of isolated amnion derived adherent cells (AMDACs), as described elsewhere herein, wherein said cells are adherent to a tissue culture surface, and wherein said cells are OCT-4⁻ (that is, negative for POU5F1, also known as octamer binding protein 4) as determinable by RT-PCR. The therapeutically effective amount is a number of AMDACs that results in elimination of, a detectable improvement in, lessening of the severity of, one or more symptoms of, schizophrenia or schizophreniform disorder. In specific embodiments, said one or more symptoms comprise one or more of one or more Schneiderian First Rank symptoms, e.g., hearing thoughts audibly; hearing voices arguing; hearing voices commenting on one's actions; delusions of being controlled by others; delusions that the individual's thoughts have been withdrawn or taken by others; delusions that thoughts have been inserted or caused by others; delusions that others can read the individual's thoughts; and/or delusional perception by the individual. In certain embodiments, said symptoms comprise one or more of disordered thoughts and/or speech; hallucinations (e.g., tactile, auditory, visual, olfactory and/or gustatory hallucinations); flat or blunted affect and/or emotion; poverty of speech (alogia); inability to experience pleasure (anhedonia); lack of desire to form relationships (asociality); and/or lack of motivation (avolition). In certain embodiments, the individual is diagnosed as schizophrenic according to either or both of Diagnostic and Statistical Manual of Mental Disorders (version DSM-IV-TR), or the World Health Organization's International Statistical Classification of Diseases and Related Health Problems (ICD-10) criteria.

The methods of treating schizophrenia provided herein apply as well to treatment of schizophreniform disorder. In schizophreniform disorder, symptoms of schizophrenia have manifested in the affected individual, but the symptoms have not been present for the six months required for a diagnosis of schizophrenia according to the DSM. In certain embodiments, therefore, provided herein is a method of treating an individual having or diagnosed as having a schizophreniform disorder, comprising administering to the individual a therapeutically effective amount of AMDACs, wherein said therapeutically effective amount is an amount effective to reduce or ameliorate one or more symptoms of schizophrenia in the individual. In certain embodiments, said one or more symptoms of schizophreniform disorder comprise one or more of delusions, hallucinations, disorganized speech (e.g., resulting from formal thought disorder), disorganized or catatonic behavior, inability to show emotion (flat affect), inability to experience pleasure (anhedonia), impaired or decreased speech (aphasia), lack of desire to form relationships (asociality), or lack of motivation (avolition).

4.1.1 Prophylaxis Against Schizophrenia

In another aspect, provided herein is a method of prophylaxis against the development of schizophrenia in an infant, comprising prenatally administering AMDACs, e.g., an effective dose of AMDACs, to an individual pregnant with the infant. In certain embodiments, the individual's immune system has been activated during pregnancy by, e.g., bacterial infection, viral infection, rickettsial infection, spirochete infection, protozoal infection, exposure to allergens, or the like. In certain embodiments, the individual who gives birth to the infant has an infection during pregnancy, e.g., a bacterial infection, viral infection, rickettsial infection, spirochete infection, or protozoal infection. In a specific embodiment, said viral infection is a viral infection during the first trimester. In another specific embodiment, said bacterial infection is a bacterial infection in the second trimester.

4.1.2 Administration

In certain embodiments, the methods of treatment of an individual having or diagnosed with schizophrenia, or schizophreniform disorder, provided herein, comprises administering to said individual between 1×10⁵ and 1×10¹¹ AMDACs. In specific embodiments, said method comprises administering about, at least, or at most, about 1×10⁵, 2.5×10⁵, 5×10⁵, 7.5×10⁵, 1×10⁶, 2.5×10⁶, 5×10⁶, 7.5×10⁶, 1×10⁷, 2.5×10⁷, 5×10⁷, 7.5×10⁷ 1×10⁸, 2.5×10⁸, 5×10⁸, 7.5×10⁸, 1×10⁹, 2.5×10⁹, 5×10⁹, 7.5×10⁹, 1×10¹⁰, 2.5×10¹⁰, 5×10¹⁰, 7.5×10¹⁰, 1×10¹¹, 2.5×10¹¹ 5×10¹¹ or more AMDACs. In specific embodiments, the method comprises administering a single unit dose of AMDACs to said individual, wherein said single unit dose comprises about 1×10⁵, 2.5×10⁵, 5×10⁵, 7.5×10⁵, 1×10⁶, 2.5×10⁶, 5×10⁶, 7.5×10⁶, 1×10⁷, 2.5×10⁷, 5×10⁷, 7.5×10⁷ 1×10⁸, 2.5×10⁸, 5×10⁸ 7.5×10⁸, 1×10⁹, 2.5×10⁹, 5×10⁹, 7.5×10⁹, 1×10¹⁰, 2.5×10¹⁰, 5×10¹⁰, 7.5×10¹⁰, 1×10¹¹, 2.5×10¹¹ 5×10¹¹ or more AMDACs. In certain other embodiments, the method comprises administering about 1×10⁵ to about 1×10⁸ AMDACs per kilogram body weight, or about 1×10⁶ to about 1×10⁷ placental stem per kilogram body weight.

In certain embodiments, the AMDACs and/or unit doses of AMDACs described above, useful for the methods of treating schizophrenia or schizophreniform disorder presented herein, are formulated into pharmaceutical compositions. Such pharmaceutical compositions can comprise AMDACs in a pharmaceutically-acceptable carrier, e.g., a saline solution or other accepted physiologically-acceptable solution for in vivo administration. Pharmaceutical compositions comprising the isolated AMDACs described herein can comprise any, or any combination, of the isolated AMDACs populations, or isolated AMDACs, described elsewhere herein. The pharmaceutical compositions provided herein can further comprise isolated AMDACs obtained from a single individual or placenta, or from a plurality of individuals or placentae. Any of the AMDACs, described elsewhere herein, can be formulated into pharmaceutical compositions, as described below.

The pharmaceutical compositions provided herein comprise at least 50% viable cells or more (that is, at least 50% of the AMDACs in the population are functional or living). Preferably, at least 60% of the cells in the population are viable. More preferably, at least 70%, 80%, 90%, 95%, or 99% of the cells in the population in the pharmaceutical composition are viable.

In certain embodiments, the pharmaceutical compositions provided herein comprise one or more compounds that, e.g., facilitate engraftment (e.g., anti-T-cell receptor antibodies, an immunosuppressant, or the like); stabilizers such as albumin, dextran 40, gelatin, hydroxyethyl starch, plasmalyte, and the like.

In certain embodiments, when formulated as an injectable solution or solution administrable by, e.g., intravenous or intraarterial infusion, the pharmaceutical composition comprises about 1% to 1.5% HSA and about 2.5% dextran. In a preferred embodiment, the pharmaceutical composition comprises from about 5×10⁶ cells (AMDACs) per mL to about 2×10⁷ cells per mL in a solution comprising 5% HSA and 10% dextran, optionally comprising an immunosuppressant, e.g., cyclosporine A at, e.g., 10 mg/kg.

In other embodiments, the pharmaceutical composition, e.g., a solution, comprises a plurality of cells, e.g., isolated AMDACs, wherein said pharmaceutical composition comprises between about 1.0±0.3×10⁶ cells per mL to about 5.0±1.5×10⁶ cells per mL. In other embodiments, the pharmaceutical composition comprises between about 1.5×10⁶ cells per mL to about 3.75×10⁶ cells per mL. In other embodiments, the pharmaceutical composition comprises between about 1×10⁶ cells/mL to about 50×10⁶ cells/mL, about 1×10⁶ cells/mL to about 40×10⁶ cells/mL, about 1×10⁶ cells/mL to about 30×10⁶ cells/mL, about 1×10⁶ cells/mL to about 20×10⁶ cells/mL, about 1×10⁶ cells/mL to about 15×10⁶ cells/mL, or about 1×10⁶ cells/mL to about 10×10⁶ cells/mL. In certain embodiments, the pharmaceutical composition comprises no visible cell clumps (i.e., no macro cell clumps), or substantially no such visible clumps. As used herein, “macro cell clumps” means an aggregation of cells visible without magnification, e.g., visible to the naked eye, and generally refers to a cell aggregation larger than about 150 microns. In some embodiments, the pharmaceutical composition comprises about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran, e.g., dextran-40. In a specific embodiment, said composition comprises about 7.5% to about 9% dextran-40. In a specific embodiment, said composition comprises about 5.5% dextran-40. In certain embodiments, the pharmaceutical composition comprises from about 1% to about 15% human serum albumin (HSA). In specific embodiments, the pharmaceutical composition comprises about 1%, 2%, 3%, 4%, 5%, 6%, 7%, 8%, 9%, 10%, 11%, 12%, 13%, 14% or 15% HSA. In a specific embodiment, said cells have been cryopreserved and thawed. In another specific embodiment, said cells have been filtered through a 70 μM to 100 μM filter. In another specific embodiment, said composition comprises no visible cell clumps. In another specific embodiment, said composition comprises fewer than about 200 cell clumps per 10⁶ cells, wherein said cell clumps are visible only under a microscope, e.g., a light microscope. In another specific embodiment, said composition comprises fewer than about 150 cell clumps per 10⁶ cells, wherein said cell clumps are visible only under a microscope, e.g., a light microscope. In another specific embodiment, said composition comprises fewer than about 100 cell clumps per 10⁶ cells, wherein said cell clumps are visible only under a microscope, e.g., a light microscope.

In a specific embodiment, the pharmaceutical composition comprises about 1.0±0.3×10⁶ cells per mL, about 5.5% dextran-40 (w/v), about 10% HSA (w/v), and about 5% DMSO (v/v).

In other embodiments, the pharmaceutical composition comprises a plurality of cells, e.g., a plurality of isolated AMDACs, in a solution comprising 10% dextran-40, wherein the pharmaceutical composition comprises between about 1.0±0.3×10⁶ cells per mL to about 5.0±1.5×10⁶ cells per mL, and wherein said composition comprises no cell clumps visible with the unaided eye (i.e., comprises no macro cell clumps). In some embodiments, the pharmaceutical composition comprises between about 1.5×10⁶ cells per mL to about 3.75×10⁶ cells per mL. In a specific embodiment, said cells have been cryopreserved and thawed. In another specific embodiment, said cells have been filtered through a 70 μM to 100 μM filter. In another specific embodiment, said composition comprises fewer than about 200 micro cell clumps (that is, cell clumps visible only with magnification) per 10⁶ cells. In another specific embodiment, the pharmaceutical composition comprises fewer than about 150 micro cell clumps per 10⁶ cells. In another specific embodiment, the pharmaceutical composition comprises fewer than about 100 micro cell clumps per 10⁶ cells. In another specific embodiment, the pharmaceutical composition comprises less than 15%, 14%, 13%, 12%, 11%, 10%, 9%, 8%, 7%, 6%, 5%, 4%, 3%, or 2% DMSO, or less than 1%, 0.9%, 0.8%, 0.7%, 0.6%, 0.5%, 0.4%, 0.3%, 0.2%, or 0.1% DMSO.

Further provided herein are compositions comprising cells, wherein said compositions are produced by one of the methods disclosed herein. For example, in one embodiment, the pharmaceutical composition comprises AMDACs, wherein the pharmaceutical composition is produced by a method comprising filtering a solution comprising AMDACs to form a filtered cell-containing solution; diluting the filtered cell-containing solution with a first solution to about 1 to 50×10⁶, 1 to 40×10⁶, 1 to 30×10⁶, 1 to 20×10⁶, 1 to 15×10⁶, or 1 to 10×10⁶ cells per mL, e.g., prior to cryopreservation; and diluting the resulting filtered cell-containing solution with a second solution comprising dextran, but not comprising human serum albumin (HSA) to produce said composition. In certain embodiments, said diluting is to no more than about 15×10⁶ cells per mL. In certain embodiments, said diluting is to no more than about 10±3×10⁶ cells per mL. In certain embodiments, said diluting is to no more than about 7.5×10⁶ cells per mL. In other certain embodiments, if the filtered cell-containing solution, prior to the dilution, comprises less than about 15×10⁶ cells per mL, filtration is optional. In other certain embodiments, if the filtered cell-containing solution, prior to the dilution, comprises less than about 10±3×10⁶ cells per mL, filtration is optional. In other certain embodiments, if the filtered cell-containing solution, prior to the dilution, comprises less than about 7.5×10⁶ cells per mL, filtration is optional.

In a specific embodiment, the AMDACs are cryopreserved between said diluting with a first dilution solution and said diluting with said second dilution solution. In another specific embodiment, the first dilution solution comprises dextran and HSA. The dextran in the first dilution solution or second dilution solution can be dextran of any molecular weight, e.g., dextran having a molecular weight of from about 10 kDa to about 150 kDa. In some embodiments, said dextran in said first dilution solution or said second solution is about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran. In another specific embodiment, the dextran in said first dilution solution or said second dilution solution is dextran-40. In another specific embodiment, the dextran in said first dilution solution and said second dilution solution is dextran-40. In another specific embodiment, said dextran-40 in said first dilution solution is 5.0% dextran-40. In another specific embodiment, said dextran-40 in said first dilution solution is 5.5% dextran-40. In another specific embodiment, said dextran-40 in said second dilution solution is 10% dextran-40. In another specific embodiment, said HSA in said solution comprising HSA is 1 to 15% HSA. In another specific embodiment, said HSA in said solution comprising HSA is about 1%, 2%, 3%, 4%, 5%, 6%, 7%, 8%, 9%, 10%, 11%, 12%, 13%, 14% or 15% HSA. In another specific embodiment, said HSA in said solution comprising HSA is 10% HSA. In another specific embodiment, said first dilution solution comprises HSA. In a more specific embodiment, said HSA in said first dilution solution is 10% HSA. In another specific embodiment, said first dilution solution comprises a cryoprotectant. In a more specific embodiment, said cryoprotectant is DMSO. In another specific embodiment, said dextran-40 in said second dilution solution is about 10% dextran-40. In another specific embodiment, said composition comprising cells comprises about 7.5% to about 9% dextran. In another specific embodiment, the pharmaceutical composition comprises from about 1.0±0.3×10⁶ cells per mL to about 5.0±1.5×10⁶ cells per mL. In another specific embodiment, the pharmaceutical composition comprises from about 1.5×10⁶ cells per mL to about 3.75×10⁶ cells per mL.

In another embodiment, the pharmaceutical composition is made by a method comprising (a) filtering a cell-containing solution comprising AMDACs prior to cryopreservation to produce a filtered cell-containing solution; (b) cryopreserving the cells in the filtered cell-containing solution at about 1 to 50×10⁶, 1 to 40×10⁶, 1 to 30×10⁶, 1 to 20×10⁶, 1 to 15×10⁶, or 1 to 10×10⁶ cells per mL; (c) thawing the cells; and (d) diluting the filtered cell-containing solution about 1:1 to about 1:11 (v/v) with a dextran-40 solution. In certain embodiments, if the number of cells is less than about 10±3×10⁶ cells per mL prior to step (a), filtration is optional. In a more specific embodiment, the cells in step (b) are cryopreserved at about 10±3×10⁶ cells per mL. In a more specific embodiment, the cells in step (b) are cryopreserved in a solution comprising about 5% to about 10% dextran-40 and HSA. In certain embodiments, said diluting in step (b) is to no more than about 15×10⁶ cells per mL.

In another embodiment, the pharmaceutical composition is made by a method comprising: (a) suspending AMDACs in a 5.5% dextran-40 solution that comprises 10% HSA to form a cell-containing solution; (b) filtering the cell-containing solution through a 70 μM filter; (c) diluting the cell-containing solution with a solution comprising 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per mL; (d) cryopreserving the cells; (e) thawing the cells; and (f) diluting the cell-containing solution 1:1 to 1:11 (v/v) with 10% dextran-40. In certain embodiments, said diluting in step (c) is to no more than about 15×10⁶ cells per mL. In certain embodiments, said diluting in step (c) is to no more than about 10±3×10⁶ cells/mL. In certain embodiments, said diluting in step (c) is to no more than about 7.5×10⁶ cells/mL.

In another embodiment, the composition comprising cells is made by a method comprising: (a) centrifuging AMDACs to collect the cells; (b) resuspending the cells in 5.5% dextran-40; (c) centrifuging the cells to collect the cells; (d) resuspending the cells in a 5.5% dextran-40 solution that comprises 10% HSA; (e) filtering the cells through a 70 μM filter; (f) diluting the cells in 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per mL; (g) cryopreserving the cells; (h) thawing the cells; and (i) diluting the cells 1:1 to 1:11 (v/v) with 10% dextran-40. In certain embodiments, said diluting in step (f) is to no more than about 15×10⁶ cells per mL. In certain embodiments, said diluting in step (f) is to no more than about 10±3×10⁶ cells/mL. In certain embodiments, said diluting in step (f) is to no more than about 7.5×10⁶ cells/mL. In other certain embodiments, if the number of cells is less than about 10±3×10⁶ cells per mL, filtration is optional.

The pharmaceutical compositions comprising the isolated AMDACs described herein can comprise any of the isolated AMDACs described herein.

Other injectable formulations, suitable for the administration of cellular products, may be used.

In certain embodiments, the AMDACs can be encapsulated in, e.g., alginate, either before or after cryopreservation. In certain other embodiments, the AMDACs can be combined with platelet-rich plasma, e.g., for local injection or local administration applications. In specific embodiments, the platelet rich plasma is autologous platelet rich plasma, e.g., autologous to the individual to whom the AMDACs are administered. In other specific embodiments, the platelet-rich plasma is allogeneic to the individual to whom the AMDACs are administered. In another specific embodiment, said platelet rich plasma is derived from placental perfusate. In other specific embodiments, the volume to volume ratio of AMDACs to platelet rich plasma in the composition, or the ratio between numbers of AMDACs and numbers of platelets, is between about 10:1 and 1:10; between about 100:1 and 1:100; or is about 1:1.

In one embodiment, the pharmaceutical composition comprises isolated AMDACs that are substantially, or completely, non-maternal in origin, that is, have the fetal genotype; e.g., at least about 90%, 95%, 98%, 99% or about 100% are non-maternal in origin.

In a specific embodiment, the pharmaceutical composition additionally comprises stem cells that are not AMDACs.

Isolated AMDACs in the compositions, e.g., pharmaceutical compositions, provided herein, can comprise AMDACs derived from a single donor, or from multiple donors. The isolated AMDACs can be completely HLA-matched to an intended recipient, or partially or completely HLA-mismatched.

4.2 Characteristics of Amnion Derived Adherent Cells

The AMDACs, useful in the method of treating schizophrenia or schizophreniform disorder provided herein, are obtainable from the amniotic membrane by a two-step isolation procedure described below, adhere to a cell culture surface, e.g., to tissue culture plastic, are OCT-4⁻ (octamer binding protein 4), as determinable by RT-PCR, and display some or all of the characteristics listed below.

AMDACs display cellular markers that distinguish them from other amnion-derived, or placenta-derived, cells. For example, in one embodiment, the OCT-4⁻ AMDACs are additionally CD49f⁺, as determinable by immunolocalization. In another specific embodiment, said AMDACs are HLA-G⁻, as determined by RT-PCR. In another specific embodiment, the OCT-4⁻ AMDACs are VEGFR1/Flt-1⁺ (vascular endothelial growth factor receptor 1) and/or VEGFR2/KDR⁺ (vascular endothelial growth factor receptor 2), as determinable by immunolocalization. In a specific embodiment, the OCT-4⁻ AMDACs express at least 2 log less PCR-amplified mRNA for OCT-4 at, e.g., 20 cycles, than an equivalent number of NTERA-2 cells for an equivalent number of RNA amplification cycles. In another specific embodiment, said OCT-4⁻ AMDACs are CD90⁺, CD105⁺, or CD117⁻. In a more specific embodiment, said OCT-4⁻ AMDACs are CD90⁺, CD105⁺, and CD117⁻, e.g., as determinable by immunolocalization. In a more specific embodiment, the AMDACs are OCT-4⁻ and/or HLA-G⁻, and are additionally CD49f⁺, CD90⁺, CD105⁺, and CD117⁻, e.g., as determinable by immunolocalization. In a more specific embodiment, the AMDACs are OCT-4⁻, HLA-G⁻, CD49f⁺, CD90⁺, CD105⁺, and CD117⁻, e.g., as determinable by immunolocalization. In another specific embodiment, the OCT-4⁻ AMDACs do not express SOX2, e.g., as determinable by RT-PCR for 30 cycles. In a specific embodiment, therefore, the cell is OCT-4⁻, CD49f⁺, CD90⁺, CD105⁺, and CD117⁻, as determinable by immunolocalization, and SOX2⁻, as determinable by RT-PCR, e.g., for 30 cycles.

In another embodiment, said OCT-4⁻ AMDACs are one or more of CD29⁺, CD73⁺, ABC-p⁺, and CD38⁻, as determined by immunolocalization.

In another specific embodiment, for example, the OCT-4⁻ AMDACs are additionally one or more of CD9⁺, CD10⁺, CD44⁺, CD54⁺, CD98⁺, TEM-7⁺ (tumor endothelial marker 7), CD31⁻, CD34⁻, CD45⁻, CD133⁻, CD143⁻ (angiotensin-I-converting enzyme, ACE), CD146⁻(melanoma cell adhesion molecule), or CXCR4⁻ (chemokine (C-X-C motif) receptor 4) as determined by immunolocalization, or HLA-G⁻ as determined by RT-PCR. In a more specific embodiment, said cell is CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determined by immunolocalization, and HLA-G⁻ as determined by RT-PCR. In one embodiment, the amnion derived adherent cell provided herein is one or more of CD31⁻, CD34⁻, CD45⁻, and/or CD133⁻. In a specific embodiment, the amnion derived adherent cell is OCT-4⁻, as determined by RT-PCR; VEGFR1/Flt-1⁺ and/or VEGFR2/KDR⁺, as determined by immunolocalization; and one or more, or all, of CD31⁻, CD34⁻, CD45⁻, and/or CD133⁻.

In another specific embodiment, said cell is additionally VE-cadherin⁻ as determined by immunolocalization. In another specific embodiment, said cell is additionally positive for CD105⁺ and CD200⁺ as determined by immunolocalization. In another specific embodiment, said cell does not express CD34 as detected by immunolocalization after exposure to 1 to 100 ng/mL VEGF for 4 to 21 days. In more specific embodiments, said cell does not express CD34 as detected by immunolocalization after exposure to 25 to 75 ng/mL VEGF for 4 to 21 days, or to 50 ng/mL VEGF for 4 to 21 days. In even more specific embodiments, said cell does not express CD34 as detected by immunolocalization after exposure to 1, 2.5, 5, 10, 25, 50, 75 or 100 ng/mL VEGF for 4 to 21 days. In yet more specific embodiments, said cell does not express CD34 as detected by immunolocalization after exposure to 1 to 100 ng/mL VEGF for 7 to 14, e.g., 7, days.

In specific embodiments, the amnion derived adherent cell is OCT-4⁻, as determined by RT-PCR, and one or more of VE-cadherin⁻, VEGFR2/KDR⁺, CD9⁺, CD54⁺, CD105⁺, and/or CD200⁺ as determined by immunolocalization. In a specific embodiment, the amnion derived cell is OCT-4⁻, as determined by RT-PCR, and VE-cadherin⁻, VEGFR2/KDR⁺, CD9⁺, CD54⁺, CD105⁺, and CD200⁺ as determined by immunolocalization. In another specific embodiment, said cells do not express CD34, as detected by immunolocalization, e.g., after exposure to 1 to 100 ng/mL VEGF for 4 to 21 days.

In another embodiment, the amnion derived adherent cell is OCT-4⁻, CD49f⁺, HLA-G⁻, CD90⁺, CD105⁺, and CD117⁺. In a more specific embodiment, said cell is one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, or CXCR4⁻, as determined by immunolocalization. In a more specific embodiment, said cell is CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determined by immunolocalization. In another specific embodiment, said cell is additionally VEGFR1/Flt-1⁺ and/or VEGFR2/KDR⁺, as determined by immunolocalization; and one or more of CD31⁻, CD34⁻, CD45⁻, CD133⁻, and/or Tie-2⁻ as determined by immunolocalization. In another specific embodiment, said cell is additionally VEGFR1/Flt-1⁺, VEGFR2/KDR⁺, CD31⁻, CD34⁻, CD45⁻, CD133⁻, and Tie-2⁻ as determined by immunolocalization.

In another embodiment, the OCT-4-amnion derived adherent cells are additionally one or more, or all, of CD9⁺, CD10⁺, CD44⁺, CD49f⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309⁺), as determined by immunolocalization; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, and/or VE-cadherin⁻, as determined by immunolocalization, or SOX2⁻, as determined by RT-PCR.

In certain embodiments, the isolated tissue culture plastic-adherent amnion derived adherent cells are CD49f⁺. In a specific embodiment, said CD49f⁺ cells are additionally one or more, or all, of CD9⁺, CD10⁺, CD44⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309′), as determined by immunolocalization; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, OCT-4⁻ and/or VE-cadherin⁻, as determined by immunolocalization, or SOX2⁻, as determined by RT-PCR.

In certain other embodiments, the isolated tissue culture plastic-adherent amnion derived adherent cells are HLA-G⁻, CD90⁺, and CD117⁻. In a specific embodiment, said HLA-G⁻, CD90⁺, and CD117⁻ cells are additionally one or more, or all, of CD9⁺, CD10⁺, CD44⁺, CD49f⁺, CD54⁺, CD98⁺, CD105⁺, CD200⁺, Tie-2⁺, TEM-7⁺, VEGFR1/Flt-1⁺, and/or VEGFR2/KDR⁺ (CD309⁺), as determined by immunolocalization; or additionally one or more, or all, of CD31⁻, CD34⁻, CD38⁻, CD45⁻, CD133⁻, CD143⁻, CD144⁻, CD146⁻, CD271⁻, CXCR4⁻, OCT-4⁻ and/or VE-cadherin⁻, as determined by immunolocalization, or SOX2⁻, as determined by RT-PCR.

In another embodiment, the isolated amnion derived adherent cells, or population of amnion derived angiogenic cells, do not constitutively express mRNA for fibroblast growth factor 4 (FGF4), interferon γ (IFNG), chemokine (C-X-C motif) ligand 10 (CXCL10), angiopoietin 4 (ANGPT4), angiopoietin-like 3 (ANGPTL3), fibrinogen a chain (FGA), leptin (LEP), prolactin (PRL), prokineticin 1 (PROK1), tenomodulin (TNMD), FMS-like tyrosine kinase 3 (FLT3), extracellular link domain containing 1 (XLKD1), cadherin 5, type 2 (CDH5), leukocyte cell derived chemotaxin 1 (LECT1), plasminogen (PLG), telomerase reverse transcriptase (TERT), (sex determining region Y)-box 2 (SOX2), NANOG, matrix metalloprotease 13 (MMP-13), distal-less homeobox 5 (DLX5), and/or bone gamma-carboxyglutamate (gla) protein (BGLAP), as determined by RT-PCR, e.g., for 30 cycles under standard culture conditions. In other embodiments, isolated amnion derived adherent cells, or population of amnion derived angiogenic cells, express mRNA for (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB1R), and hepatoycte growth factor receptor (HGFR/c-met)

In another aspect, provided herein are isolated populations of cells comprising the amnion derived adherent cells described herein. The populations of cells can be homogeneous populations, e.g., a population of cells, at least about 90%, 95%, 98% or 99% of which are amnion derived adherent cells. The populations of cells can be heterogeneous, e.g., a population of cells wherein at most about 10%, 20%, 30%, 40%, 50%, 60%, 70% or 80% of the cells in the population are amnion derived adherent cells. The isolated populations of cells are not, however, tissue, i.e., amniotic membrane.

In one embodiment, provided herein is an isolated population of cells comprising AMDACs, e.g., a population of cells substantially homogeneous for AMDACs, wherein said AMDACs are adherent to tissue culture plastic, and wherein said AMDACs are OCT-4⁻, as determined by RT-PCR. In a specific embodiment, the AMDACs are CD49f⁺ or HLA-G⁺, e.g., as determined by immunolocalization or RT-PCR. In another specific embodiment, said population of AMDACs is VEGFR1/Flt-1⁺ and/or VEGFR2/KDR⁺ as determined by immunolocalization, wherein said isolated population of cells is not an amnion or amniotic membrane. In a more specific embodiment, the AMDACs are OCT-4⁻, and/or HLA-G⁻ as determined by RT-PCR, and VEGFR1/Flt-1⁺ and/or VEGFR2/KDR⁺ as determined by immunolocalization. In a specific embodiment, at least about 50%, 60%, 70%, 80%, 90%, 95%, 98% or 99% of cells in said population are said amnion derived adherent cells. In another specific embodiment, said AMDACs are CD90⁺, CD105⁺, or CD117⁺. In a more specific embodiment, said AMDACs are CD90⁺, CD105⁺, and CD117⁻. In a more specific embodiment, the AMDACs are OCT-4⁻, CD49f⁺, CD90⁺, CD105⁺, and CD117⁻. In another specific embodiment, the AMDACs do not express SOX2, e.g., as determined by RT-PCR for 30 cycles. In an even more specific embodiment, the population comprises AMDACs, wherein said AMDACs are OCT-4⁻, HLA-G⁻, CD49f⁺, CD90⁺, CD105⁺, and CD117⁻, as determined by immunolocalization or flow cytometry, and SOX2⁻, e.g., as determined by RT-PCR for 30 cycles

In another specific embodiment, said AMDACs in said population of cells are CD90⁺, CD105⁺, or CD117⁻, as determined by immunolocalization or flow cytometry. In a more specific embodiment, the AMDACs are CD90⁺, CD105⁺, and CD117⁻, as determined by immunolocalization or flow cytometry. In a more specific embodiment, the AMDACs are OCT-4⁻ or HLA-G⁻, e.g., as determined by RT-PCR, and are additionally CD49f⁺, CD90⁺, CD105⁺, and CD117⁻ as determined by immunolocalization or flow cytometry. In a more specific embodiment, the AMDACs in said population of cells are OCT-4⁻, HLA-G⁻, CD49f⁺, CD90⁺, CD105⁺, and CD117⁻. In another specific embodiment, the AMDACs do not express SOX2, e.g., as determined by RT-PCR for 30 cycles. In a more specific embodiment, therefore, the cell is OCT-4⁻, CD49f⁺, CD90⁺, CD105⁺, and CD117⁻, as determined by immunolocalization or flow cytometry, and SOX2⁻, as determined by RT-PCR, e.g., for 30 cycles. In an even more specific embodiment, the AMDACs are OCT-4⁻ or HLA-G⁻, and are additionally CD49f⁺, CD90⁺, CD105⁺, and CD117⁻. In a more specific embodiment, the AMDACs are OCT-4⁻, HLA-G⁻, CD49f⁺, CD90⁺, CD105⁺, and CD117⁻.

In another embodiment, the amnion derived adherent cells in said population of cells are adherent to tissue culture plastic, OCT-4⁻ as determined by RT-PCR, and VEGFR1/Flt-1⁺ and/or VEGFR2/KDR⁺ as determined by immunolocalization, and are additionally one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, or CXCR4⁻, as determined by immunolocalization, or HLA-G⁻ as determined by RT-PCR, and wherein said isolated population of cells is not an amnion. In another embodiment, provided herein is an isolated population of cells comprising an amnion derived adherent cell, wherein said cell is adherent to tissue culture plastic, wherein said cell is OCT-4⁻ as determined by RT-PCR, and VEGFR1/Flt-1⁺ and/or VEGFR2/KDR⁺ as determined by immunolocalization, wherein said cell does not express CD34 as detected by immunolocalization after exposure to 1 to 100 ng/mL VEGF for 4 to 21 days, and wherein said isolated population of cells is not an amnion. In a specific embodiment of any of the above embodiments, at least about 50%, 60%, 70%, 80%, 90%, 95%, 98% or 99% of cells in said population are said amnion derived adherent cells.

In another embodiment, any of the above populations of cells comprising amnion derived adherent cells forms sprouts or tube-like structures when cultured in the presence of an extracellular matrix protein, e.g., like collagen type I and IV, or an angiogenic factor, e.g., like vascular endothelial growth factor (VEGF), epithelial growth factor (EGF), platelet derived growth factor (PDGF) or basic fibroblast growth factor (bFGF), e.g., in or on a substrate such as placental collagen, e.g., or MATRIGEL™ for at least 4 days and up to 14 days.

Amnion derived adherent cells, and populations of amnion derived adherent cells, display characteristic expression of proteins related to angiogenesis-related or cardiomyogenesis-related genes. In certain embodiments, provided herein is a cell that expresses, or a population of cells, wherein at least about 50%, 60%, 70%, 80%, 90%, 95% or 98% of cells in said isolated population of cells are amnion derived adherent cells that express RNA for one or more of, or all of, ACTA2 (actin, alpha 2, smooth muscle, aorta), ADAMTS1 (ADAM metallopeptidase with thrombospondin type 1 motif, 1), AMOT (angiomotin), ANG (angiogenin), ANGPT1 (angiopoietin 1), ANGPT2, ANGPTL1 (angiopoietin-like 1), ANGPTL2, ANGPTL4, BAI1 (brain-specific angiogenesis inhibitor 1), CD44, CD200, CEACAM1 (carcinoembryonic antigen-related cell adhesion molecule 1), CHGA (chromogranin A), COL15A1 (collagen, type XV, alpha 1), COL18A1 (collagen, type XVIII, alpha 1), COL4A1 (collagen, type IV, alpha 1), COL4A2 (collagen, type IV, alpha 2), COL4A3 (collagen, type IV, alpha 3), CSF3 (colony stimulating factor 3 (granulocyte), CTGF (connective tissue growth factor), CXCL12 (chemokine (CXC motif) ligand 12 (stromal cell-derived factor 1)), CXCL2, DNMT3B (DNA (cytosine-5-)-methyltransferase 3 beta), ECGF1 (thymidine phosphorylase), EDG1 (endothelial cell differentiation gene 1), EDIL3 (EGF-like repeats and discoidin I-like domains 3), ENPP2 (ectonucleotide pyrophosphatase/phosphodiesterase 2), EPHB2 (EPH receptor B2), FBLN5 (FIBULIN 5), F2 (coagulation factor II (thrombin)), FGF1 (acidic fibroblast growth factor), FGF2 (basic fibroblast growth factor), FIGF (c-fos induced growth factor (vascular endothelial growth factor D)), FLT4 (fms-related tyrosine kinase 4), FN1 (fibronectin 1), FST (follistatin), FOXC2 (forkhead box C2 (MFH-1, mesenchyme forkhead 1)), GRN (granulin), HGF (hepatocyte growth factor), HEY1 (hairy/enhancer-of-split related with YRPW motif 1), HSPG2 (heparan sulfate proteoglycan 2), IFNB1 (interferon, beta 1, fibroblast), IL8 (interleukin 8), IL12A, ITGA4 (integrin, alpha 4; CD49d), ITGAV (integrin, alpha V), ITGB3 (integrin, beta 3), MDK (midkine), MMP2 (matrix metalloprotease 2), MYOZ2 (myozenin 2), NRP1 (neuropilin 1), NRP2, PDGFB (platelet-derived growth factor β), PDGFRA (platelet-derived growth factor receptor α), PDGFRB, PECAM1 (platelet/endothelial cell adhesion molecule), PF4 (platelet factor 4), PGK1 (phosphoglycerate kinase 1), PROX1 (prospero homeobox 1), PTN (pleiotrophin), SEMA3F (semophorin 3F), SERPINB5 (serpin peptidase inhibitor, clade B (ovalbumin), member 5), SERPINC1, SERPINF1, TIMP2 (tissue inhibitor of metalloproteinases 2), TIMP3, TGFA (transforming growth factor, alpha), TGFB1, THBS1 (thrombospondin 1), THBS2, TIE1 (tyrosine kinase with immunoglobulin-like and EGF-like domains 1), TIE2/TEK, TNF (tumor necrosis factor), TNNI1 (troponin I, type 1), TNFSF15 (tumor necrosis factor (ligand) superfamily, member 15), VASH1 (vasohibin 1), VEGF (vascular endothelial growth factor), VEGFB, VEGFC, VEGFR1/FLT1 (vascular endothelial growth factor receptor 1), and/or VEGFR2/KDR.

When human cells are used, the gene designations throughout refer to human sequences, and, as is well known to persons of skill in the art, representative sequences can be found in literature, or in GenBank. Probes to the sequences can be determined by sequences that are publicly-available, or through commercial sources, e.g., specific TAQMAN® probes or TAQMAN® Angiogenesis Array (Applied Biosystems, part no. 4378710).

Amnion derived adherent cells, and populations of amnion derived adherent cells, display characteristic expression of angiogenesis-related proteins. In certain embodiments, provided herein is a cell that expresses, or a population of cells, wherein at least about 50%, 60%, 70%, 80%, 90%, 95% or 98% of cells in said isolated population of cells are amnion derived adherent cells that express CD49d, Connexin-43, HLA-ABC, Beta 2-microglobulin, CD349, CD318, PDL1, CD106, Galectin-1, ADAM 17 precursor (A disintegrin and metalloproteinase domain 17) (TNF-alpha converting enzyme) (TNF-alpha convertase), Angiotensinogen precursor, Filamin A (Alpha-filamin) (Filamin 1) (Endothelial actin-binding protein) (ABP-280) (Nonmuscle filamin), Alpha-actinin 1 (Alpha-actinin cytoskeletal isoform) (Non-muscle alpha-actinin 1) (F-actin cross linking protein), Low-density lipoprotein receptor-related protein 2 precursor (Megalin) (Glycoprotein 330) (gp330), Macrophage scavenger receptor types I and II (Macrophage acetylated LDL receptor I and II), Activin receptor type JIB precursor (ACTR-IIB), Wnt-9 protein, Glial fibrillary acidic protein, astrocyte (GFAP), Myosin-binding protein C, cardiac-type (Cardiac MyBP-C) (C-protein, cardiac muscle isoform), and/or Myosin heavy chain, nonmuscle type A (Cellular myosin heavy chain, type A) (Nonmuscle myosin heavy chain-A) (NMMHC-A).

The amnion derived adherent cells provided herein further secrete proteins that promote angiogenesis, e.g., in endothelial cells, endothelial progenitor cells, or the like. In certain embodiments, the amnion derived adherent cell, population of amnion derived adherent cells, or population of cells comprising amnion derived adherent cells, e.g., wherein at least about 50%, 60%, 70%, 80%, 90%, 95% or 98% of cells in said isolated population of cells are amnion derived adherent cells, secrete one or more, or all, of VEGF, HGF, IL-8, MCP-3, FGF2, Follistatin, G-CSF, EGF, ENA-78, GRO, IL-6, MCP-1, PDGF-BB, TIMP-2, uPAR, Galectin-1, e.g., into culture medium in which the cell, or cells, are grown.

In another embodiment, any of the above populations of cells comprising amnion derived adherent cells can cause the formation of sprouts or tube-like structures in a population of endothelial cells in contact with said amnion derived adherent cells. In a specific embodiment, the amnion-derived angiogenic cells are co-cultured with human endothelial cells, forming sprouts or tube-like structures, or supporting the endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ for at least 4 days and/or up to 14 days.

In another embodiment, any of the above populations of cells comprising amnion derived adherent cells secrete angiogenic factors such as vascular endothelial growth factor (VEGF), epithelial growth factor (EGF), platelet derived growth factor (PDGF), basic fibroblast growth factor (bFGF), or Interleukin-8 (IL-8) and thereby can induce human endothelial cells to form sprouts or tube-like structures when cultured in the presence of extracellular matrix proteins such as collagen type I and IV e.g., in or on a substrate such as placental collagen or MATRIGEL™.

In another embodiment, provided herein is a population of cells, e.g., a population of amnion derived adherent cells, or a population of cells wherein at least about 50%, 60%, 70%, 80%, 90%, 95% or 98% of cells in said isolated population of cells are amnion derived adherent cells that express angiogenic micro RNAs (miRNAs) at a higher level than bone marrow-derived mesenchymal stem cells, wherein said miRNAs comprise one or more, or all of, miR-17-3p, miR-18a, miR-18b, miR-19b, miR-92, and/or miR-296. In another embodiment, provided herein is a population of cells, e.g., a population of amnion derived adherent cells, or a population of cells wherein at least about 50%, 60%, 70%, 80%, 90%, 95% or 98% of cells in said isolated population of cells are amnion derived adherent cells that express one or more of, or all of, angiogenic micro RNAs (miRNAs) at a lower level than bone marrow-derived mesenchymal stem cells, wherein said miRNAs comprise one or more, or all of, miR-20a, miR-20b, miR-221, miR-222, miR-15b, and/or miR-16. In certain embodiments, AMDACs, or populations of AMDACs, express one or more, or all, of the angiogenic miRNAs miR-17-3p, miR-18a, miR-18b, miR-19b, miR-92, miR-20a, miR-20b, (members of the of the angiogenic miRNA cluster 17-92), miR-296, miR-221, miR-222, miR-15b, and/or miR-16.

Thus, in one embodiment, provided herein is an isolated amnion derived adherent cell, wherein said cell is adherent to tissue culture plastic, and wherein said cell is OCT-4⁻, as determined by RT-PCR, and CD49f⁺, HLA-G⁻, CD90⁺, CD105⁺, and CD117⁻, as determined by immunolocalization, and wherein said cell: (a) expresses one or more of CD9, CD10, CD44, CD54, CD98, CD200, Tie-2, TEM-7, VEGFR1/Flt-1, or VEGFR2/KDR (CD309), as determined by immunolocalization; (b) lacks expression of CD31, CD34, CD38, CD45, CD133, CD143, CD144, CD146, CD271, CXCR4, HLA-G, or VE-cadherin, as determined by immunolocalization, or lacks expression of SOX2, as determined by RT-PCR; (c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR; (d) expresses one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type JIB precursor, Wnt-9 protein, glial fibrillary acidic protein, astrocyte, myosin-binding protein C, or myosin heavy chain, nonmuscle type A; (e) secretes VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the cell grows; (f) expresses micro RNAs miR-17-3p, miR-18a, miR-18b, miR-19b, miR-92, or miR-296 at a higher level than an equivalent number of bone marrow-derived mesenchymal stem cells; (g) expresses micro RNAs miR-20a, miR-20b, miR-221, miR-222, miR-15b, or miR-16 at a lower level than an equivalent number of bone marrow-derived mesenchymal stem cells; (h) expresses miRNAs miR-17-3p, miR-18a, miR-18b, miR-19b, miR-92, miR-20a, miR-20b, miR-296, miR-221, miR-222, miR-15b, or miR-16; and/or (i) expresses increased levels of CD202b, IL-8 or VEGF when cultured in less than about 5% O₂, compared to expression of CD202b, IL-8 or VEGF under 21% O₂. In a specific embodiment, the isolated amnion derived adherent cell is OCT-4⁻, as determined by RT-PCR, and CD49f⁺, HLA-G⁻, CD90⁺, CD105⁺, and CD117⁻, as determined by immunolocalization, and (a) expresses CD9, CD10, CD44, CD54, CD90, CD98, CD200, Tie-2, TEM-7, VEGFR1/Flt-1, and/or VEGFR2/KDR (CD309), as determined by immunolocalization; (b) lacks expression of CD31, CD34, CD38, CD45, CD133, CD143, CD144, CD146, CD271, CXCR4, HLA-G, and/or VE-cadherin, as determined by immunolocalization, or lacks expression of SOX2, as determined by RT-PCR; (c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, and/or VEGFR2/KDR; (d) expresses one or more of CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A; (e) secretes VEGF, HGF, IL-8, MCP-3, FGF2, Follistatin, G-CSF, EGF, ENA-78, GRO, IL-6, MCP-1, PDGF-BB, TIMP-2, uPAR, and/or Galectin-1, e.g., into culture medium in which the cell grows; (f) expresses micro RNAs miR-17-3p, miR-18a, miR-18b, miR-19b, miR-92, and/or miR-296 at a higher level than an equivalent number of bone marrow-derived mesenchymal stem cells; (g) expresses micro RNAs miR-20a, miR-20b, miR-221, miR-222, miR-15b, and/or miR-16 at a lower level than an equivalent number of bone marrow-derived mesenchymal stem cells; (h) expresses miRNAs miR-17-3p, miR-18a, miR-18b, miR-19b, miR-92, miR-20a, miR-20b, miR-296, miR-221, miR-222, miR-15b, and/or miR-16; and/or (i) expresses increased levels of CD202b, IL-8 and/or VEGF when cultured in less than about 5% O₂, compared to expression of CD202b, IL-8 and/or VEGF under 21% O₂. Further provided herein are populations of cells comprising AMDACs, e.g. populations of AMDACs, having one or more of the above-recited characteristics.

In another embodiment, any of the above populations of cells comprising amnion derived adherent cells secretes angiogenic factors. In specific embodiments, the population of cells secretes vascular endothelial growth factor (VEGF), epithelial growth factor (EGF), platelet derived growth factor (PDGF), basic fibroblast growth factor (bFGF), and/or interleukin-8 (IL-8). In other specific embodiments, the population of cells comprising amnion-derived angiogenic cells secretes one or more angiogenic factors and thereby induces human endothelial cells to migrate in an in vitro wound healing assay. In other specific embodiments, the population of cells comprising amnion derived adherent cells induces maturation, differentiation or proliferation of human endothelial cells, endothelial progenitors, myocytes or myoblasts.

In another embodiment, any of the above populations of cells comprising amnion derived adherent cells take up acetylated low density lipoprotein (LDL) when cultured in the presence of extracellular matrix proteins, e.g., collagen type I or IV, and/or one or more angiogenic factors, e.g., VEGF, EGF, PDGF, or bFGF, e.g., on a substrate such as placental collagen or MATRIGEL™.

In another embodiment, provided herein is a population of cells comprising amnion derived adherent cells, wherein said cells are adherent to tissue culture plastic, and wherein said cells are OCT-4⁻, as determined by RT-PCR, and VEGFR2/KDR⁺, CD9⁺, CD54⁺, CD105⁺, CD200⁺, or VE-cadherin⁻, as determined by immunolocalization. In specific embodiments, at least 10%, 20%, 30%, 40%, 50%, 60%, 70%, 80%, 90%, 95%, 98% or 99% of the cells in said population of cells are amnion derived cells that are OCT-4⁻, as determined by RT-PCR, and VEGFR2/KDR⁺, CD9⁺, CD54⁺, CD105⁺, CD200⁺, or VE-cadherin⁻, as determined by immunolocalization. In another specific embodiment, at least 10%, 20%, 30%, 40%, 50%, 60%, 70%, 80%, 90%, 95%, 98% or 99% of the cells in said population are amnion derived cells that are OCT-4⁻, as determined by RT-PCR, and VEGFR2/KDR⁺, CD9⁺, CD54⁺, CD105⁺, CD200⁺, and VE-cadherin⁻, as determined by immunolocalization. In another specific embodiment, said cells that are OCT-4⁻, as determined by RT-PCR, and VEGFR2/KDR⁺, CD9⁺, CD54⁺, CD105⁺, CD200⁺, or VE-cadherin⁻, as determined by immunolocalization, do not express CD34, as detected by immunolocalization, after exposure to 1 to 100 ng/mL VEGF for 4 to 21 days. In another specific embodiment, said cells are also VE-cadherin⁻.

The populations of cells provided herein, comprising amnion derived adherent cells, are able to form sprouts or tube-like structures resembling vessels or vasculature. In one embodiment, the populations of cells comprising amnion derived adherent cells form sprouts or tube-like structures when cultured in the presence of an angiogenic moiety, e.g., VEGF, EGF, PDGF or bFGF. In a more specific embodiment, said amnion derived cells that are OCT-4⁻, as determined by RT-PCR, and VEGFR2/KDR⁺, CD9⁺, CD54⁺, CD105⁺, CD200⁺, or VE-cadherin⁻, as determined by immunolocalization, form sprouts or tube-like structures when said population of cells is cultured in the presence of vascular endothelial growth factor (VEGF).

The amnion derived adherent cells described herein display the above characteristics, e.g., combinations of cell surface markers and/or gene expression profiles, and/or angiogenic potency and function, in primary culture, or during proliferation in medium suitable for the culture of stem cells. Such medium includes, for example, medium comprising 1 to 100% DMEM-LG (Gibco), 1 to 100% MCDB-201(Sigma), 1 to 10% fetal calf serum (FCS) (Hyclone Laboratories), 0.1 to 5× insulin-transferrin-selenium (ITS, Sigma), 0.1 to 5× linolenic-acid-bovine-serum-albumin (LA-BSA, Sigma), 10⁻⁵ to 10⁻¹⁵M dexamethasone (Sigma), 10⁻² to 10⁻¹⁰ M ascorbic acid 2-phosphate (Sigma), 1 to 50 ng/mL epidermal growth factor (EGF), (R&D Systems), 1 to 50 ng/mL platelet derived-growth factor (PDGF-BB) (R&D Systems), and 100U penicillin/1000U streptomycin. In a specific embodiment, the medium comprises 60% DMEM-LG (Gibco), 40% MCDB-201(Sigma), 2% fetal calf serum (FCS) (Hyclone Laboratories), 1× insulin-transferrin-selenium (ITS), 1× linolenic-acid-bovine-serum-albumin (LA-BSA), 10⁻⁹M dexamethasone (Sigma), 10⁻⁴M ascorbic acid 2-phosphate (Sigma), epidermal growth factor (EGF)10 ng/ml (R&D Systems), platelet derived-growth factor (PDGF-BB) 10 ng/ml (R&D Systems), and 100U penicillin/1000U streptomycin Other suitable media are described below.

The isolated populations of amnion derived adherent cells provided herein can comprise about, at least about, or no more than about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more amnion derived adherent cells, e.g., in a container. In various embodiments, at least 10%, 20%, 30%, 40%, 50%, 60%, 70%, 80%, 90%, 95%, or 99% of the cells in the isolated cell populations provided herein are amnion derived adherent cells. That is, a population of isolated amnion derived adherent cells can comprise, e.g., as much as 1%, 5%, 10%, 20%, 30%, 40%, 50%, 60%, 70%, 80%, 90% non-stem cells.

The amnion derived adherent cells provided herein can be cultured on a substrate. In various embodiments, the substrate can be any surface on which culture and/or selection of amnion derived adherent cells, can be accomplished. Typically, the substrate is plastic, e.g., tissue culture dish or multiwell plate plastic. Tissue culture plastic can be treated, coated or imprinted with a biomolecule or synthetic mimetic agent, e.g., CELLSTART™, MESENCULT™ ACF-substrate, ornithine, or polylysine, or an extracellular matrix protein, e.g., collagen, laminin, fibronectin, vitronectin, or the like.

Amnion derived cells, e.g., the amnion derived adherent cells provided herein, and populations of such cells, can be isolated from one or more placentas. For example, an isolated population of the amnion derived cells provided herein can be a population of placental cells comprising such cells obtained from, or contained within, disrupted amnion tissue, e.g., tissue digestate (that is, the collection of cells obtained by enzymatic digestion of an amnion), wherein said population of cells is enriched for the amnion derived cells, and wherein the tissue is from a single placenta or from two or more placentas. Isolated amnion derived cells can be cultured and expanded to produce populations of such cells. Populations of placental cells comprising amnion derived adherent cells can also be cultured and expanded to produce populations of amnion derived adherent cells.

In certain embodiments, AMDACs displaying any of the above marker and/or gene expression characteristics have been passaged at least 1, 2, 3, 4, 5, 6, 7, 8, 9, 10, 11, 12, 13, 14, 15, 16, 17, 18, 19 or 20 times, or more. In certain other embodiments, AMDACs displaying any of the above marker and/or gene expression characteristics have been doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

4.3 Populations of Amnion Derived Adherent Cells Comprising Other Cell Types

The isolated cell populations comprising amnion derived adherent cells described herein can comprise a second cell type, e.g., placental cells that are not amnion derived adherent cells, or, e.g., cells that are not placental cells. For example, an isolated population of amnion derived adherent cells can comprise, e.g., can be combined with, a population of a second type of cells, wherein said second type of cell are, e.g., embryonic stem cells, blood cells (e.g., placental blood, placental blood cells, umbilical cord blood, umbilical cord blood cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), AMDACs (e.g., the AMDACs described in U.S. Pat. No. 7,468,276, and in U.S. Patent Application Publication No. and 2007/0275362, the disclosures of which are incorporated herein by reference in their entireties), nucleated cells from placental perfusate, e.g., total nucleated cells from placental perfusate; umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, and the like. In a specific embodiment, a population of cells comprising amnion derived adherent cells comprises placental stem cells or stem cells from umbilical cord. In certain embodiments in which the second type of cell is blood or blood cells, erythrocytes have been removed from the population of cells.

In a specific embodiment, the second type of cell is a hematopoietic stem cell. Such hematopoietic stem cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

In another embodiment, an isolated population of amnion derived adherent cells is combined with a plurality of adult or progenitor cells from the vascular system. In various embodiments, the cells are endothelial cells, endothelial progenitor cells, myocytes, cardiomyocytes, pericytes, angioblasts, myoblasts or cardiomyoblasts.

In a another embodiment, the second cell type is a non-embryonic cell type manipulated in culture in order to express markers of pluripotency and functions associated with embryonic stem cells

In specific embodiments of the above isolated populations of amnion derived adherent cells, either or both of the amnion derived adherent cells and cells of a second type are autologous, or are allogeneic, to an intended recipient of the cells.

Further provided herein is a composition comprising amnion derived adherent cells, and a plurality of stem cells other than the amnion derived adherent cells. In a specific embodiment, the composition comprises stem cells that are obtained from a placenta, i.e., placental stem cells, e.g., as described in U.S. Pat. Nos. 7,045,148; 7,255,879; and 7,311,905, and in U.S. Patent Application Publication No. 2007/0275362, the disclosures of each of which are incorporated herein by reference in their entireties. In specific embodiments, said placental stem cells are CD200⁺ and HLA-G⁺; CD73⁺, CD105⁺, and CD200⁺; CD200⁺ and OCT-4⁺; CD73⁺, CD105⁺ and HLA-G⁺; CD73⁺ and CD105⁺ and facilitate the formation of one or more embryoid-like bodies in a population of placental cells comprising said stem cell when said population is cultured under conditions that allow the formation of an embryoid-like body; or OCT-4⁺ and facilitate the formation of one or more embryoid-like bodies in a population of placental cells comprising the stem cell when said population is cultured under conditions that allow formation of embryoid-like bodies; or any combination thereof. In a more specific embodiment, said CD200⁺, HLA-G⁺ stem cells are CD34⁻, CD38⁻, CD45⁻, CD73⁺ and CD105⁺. In another more specific embodiment, said CD73⁺, CD105⁺, and CD200⁺ stem cells are CD34⁻, CD38⁻, CD45⁻, and HLA-G′. In another more specific embodiment, said CD200⁺, OCT-4⁺ stem cells are CD34⁻, CD38⁻, CD45⁻, CD73⁺, CD105⁺ and HLA-G⁺. In another more specific embodiment, said CD73⁺, CD105⁺ and HLA-G⁺ stem cells are CD34⁻, CD45⁻, OCT-4⁺ and CD200⁺. In another more specific embodiment, said CD73⁺ and CD105⁺ stem cells are OCT-4⁺, CD34⁻, CD38⁻and CD45⁻. In another more specific embodiment, said OCT-4⁺ stem cells are CD73⁺, CD105⁺, CD200⁺, CD34⁻, CD38⁻, and CD45⁻. In another more specific embodiment, the placental stem cells are maternal in origin (that is, have the maternal genotype). In another more specific embodiment, the placental stem cells are fetal in origin (that is, have the fetal genotype).

In another specific embodiment, the composition comprises amnion derived adherent cells, and embryonic stem cells. In another specific embodiment, the composition comprises amnion derived adherent cells and mesenchymal stromal or stem cells, e.g., bone marrow-derived mesenchymal stromal or stem cells. In another specific embodiment, the composition comprises bone marrow-derived hematopoietic stem cells. In another specific embodiment, the composition comprises amnion derived adherent cells and hematopoietic progenitor cells, e.g., hematopoietic progenitor cells from bone marrow, fetal blood, umbilical cord blood, placental blood, and/or peripheral blood. In another specific embodiment, the composition comprises amnion derived adherent cells and somatic stem cells. In a more specific embodiment, said somatic stem cell is a neural stem cell, a hepatic stem cell, a pancreatic stem cell, an endothelial stem cell, a cardiac stem cell, or a muscle stem cell.

In other specific embodiments, the second type of cells comprise about, at least, or no more than, 10%, 15%, 20%, 25%, 30%, 35%, 40%, 45%, or 50% of cells in said population. In other specific embodiments, the AMDACs in said composition comprise at least 50%, 55%, 60%, 65%, 70%, 75%, 80%, 85% or 90% of cells in said composition. In other specific embodiments, the amnion derived adherent cells comprise about, at least, or no more than, 10%, 15%, 20%, 25%, 30%, 35%, 40%, or 45% of cells in said population.

Cells in an isolated population of amnion derived adherent cells can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated population of amnion derived adherent cells can be combined with a plurality of cells of a plurality of cell types, as well.

4.4 Growth in Culture

The growth of the amnion derived adherent cells described herein, as for any mammalian cell, depends in part upon the particular medium selected for growth. Under optimum conditions, amnion derived adherent cells typically double in number in approximately 24 hours. During culture, the amnion derived adherent cells described herein adhere to a substrate in culture, e.g. the surface of a tissue culture container (e.g., tissue culture dish plastic, fibronectin-coated plastic, and the like) and form a monolayer. Typically, the cells establish in culture within 2-7 days after digestion of the amnion. They proliferate at approximately 0.4 to 1.2 population doublings per day and can undergo at least 30 to 50 population doublings. The cells display a mesenchymal/fibroblastic cell-like phenotype during subconfluence and expansion, and a cuboidal/cobblestone-like appearance at confluence, and proliferation in culture is strongly contact-inhibited. Populations of amnion-derived angiogenic cells can form embryoid bodies during expansion in culture.

4.5 Methods of Obtaining Amnion-Derived Angiogenic Cells

The amnion derived adherent cells, and populations of cells comprising the amnion derived adherent cells, can be produced, e.g., isolated from other cells or cell populations, for example, through particular methods of digestion of amnion tissue, optionally followed by assessment of the resulting cells or cell population for the presence or absence of markers, or combinations of markers, characteristics of amnion derived adherent cells, or by obtaining amnion cells and selecting on the basis of markers characteristic of amnion derived adherent cells.

The amnion derived adherent cells, and isolated populations of cells comprising the amnion derived adherent cells, provided herein can be produced by, e.g., digestion of amnion tissue followed by selection for adherent cells. In one embodiment, for instance, isolated amnion derived adherent cells, or an isolated population of cells comprising amnion derived adherent cells, can be produced by (1) digesting amnion tissue with a first enzyme to dissociate cells from the epithelial layer of the amnion from cells from the mesenchymal layer of the amnion; (2) subsequently digesting the mesenchymal layer of the amnion with a second enzyme to form a single-cell suspension; (3) culturing cells in said single-cell suspension on a tissue culture surface, e.g., tissue culture plastic; and (4) selecting cells that adhere to said surface after a change of medium, thereby producing an isolated population of cells comprising amnion derived adherent cells. In a specific embodiment, said first enzyme is trypsin. In a more specific embodiment, said trypsin is used at a concentration of 0.25% trypsin (w/v), in 5-20, e.g., 10 milliliters solution per gram of amnion tissue to be digested. In another more specific embodiment, said digesting with trypsin is allowed to proceed for about 15 minutes at 37° C. and is repeated up to three times. In another specific embodiment, said second enzyme is collagenase. In a more specific embodiment, said collagenase is used at a concentration between 50 and 500 U/L in 5 mL per gram of amnion tissue to be digested. In another more specific embodiment, said digesting with collagenase is allowed to proceed for about 45-60 minutes at 37° C. In another specific embodiment, the single-cell suspension formed after digestion with collagenase is filtered through, e.g., a 75 μM-150 μM filter between step (2) and step (3). In another specific embodiment, said first enzyme is trypsin, and said second enzyme is collagenase.

An isolated population of cells comprising amnion derived adherent cells can, in another embodiment, be obtained by selecting cells from amnion, e.g., cells obtained by digesting amnion tissue as described elsewhere herein, that display one or more characteristics of an amnion derived adherent cell. In one embodiment, for example, a cell population is produced by a method comprising selecting amnion cells that are (a) negative for OCT-4, as determined by RT-PCR, and (b) positive for one or more of VEGFR2/KDR, CD9, CD54, CD105, CD200, as determined by immunolocalization; and isolating said cells from other cells to form a cell population. In a specific embodiment, said amnion cells are additionally VE-cadherin⁻. In a specific embodiment, a cell population is produced by selecting placental cells that are (a) negative for OCT-4, as determined by RT-PCR, and VE-cadherin, as determined by immunolocalization, and (b) positive for each of VEGFR2/KDR, CD9, CD54, CD105, CD200, as determined by immunolocalization; and isolating said cells from other cells to form a cell population. In certain embodiments, selection by immunolocalization is performed before selection by RT-PCR. In another specific embodiment, said selecting comprises selecting cells that do not express cellular marker CD34 after culture for 4 to 21 days in the presence of 1 to 100 ng/mL VEGF.

In another embodiment, for example, a cell population is produced by a method comprising selecting amnion cells that are adherent to tissue culture plastic and are OCT-4⁻, as determined by RT-PCR, and VEGFR1/Flt-1⁺ and VEGFR2/KDR⁺, as determined by immunolocalization, and isolating said cells from other cells to form a cell population. In a specific embodiment, a cell population is produced by a method comprising selecting amnion cells that are OCT-4⁻, as determined by RT-PCR, and VEGFR1/Flt-1⁺, VEGFR2/KDR⁺, and HLA-G⁻, as determined by immunolocalization. In another specific embodiment, said cell population is produced by selecting amnion cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and/or CXCR4⁻ (chemokine (C-X-C motif) receptor 4) as determined by immunolocalization, and isolating the cells from cells that do not display one or more of these characteristics. In another specific embodiment, said cell population is produced by selecting amnion cells that are additionally VE-cadherin⁻ as determined by immunolocalization, and isolating the cells from cells that are VE-cadherin⁺. In another specific embodiment, said cell population is produced by selecting amnion cells that are additionally CD105⁺ and CD200⁺ as determined by immunolocalization, and isolating the cells from cells that are CD105⁻ or CD200⁻. In another specific embodiment, said cell does not express CD34 as detected by immunolocalization after exposure to 1 to 100 ng/mL VEGF for 4 to 21 days.

In the selection of cells, it is not necessary to test an entire population of cells for characteristics specific to amnion derived adherent cells. Instead, one or more aliquots of cells (e.g., about 0.5%-2%) of a population of cells may be tested for such characteristics, and the results can be attributed to the remaining cells in the population.

Selected cells can be confirmed to be the amnion derived adherent cells provided herein by culturing a sample of the cells (e.g., about 10⁴ to about 10⁵ cells) on a substrate, e.g., MATRIGEL™, for 4 to 14, e.g., 7, days in the presence of VEGF (e.g., about 50 ng/mL), and visually inspecting the cells for the appearance of sprouts and/or cellular networks.

Amnion derived adherent cells can be selected by the above markers using any method known in the art of cell selection. For example, the adherent cells can be selected using an antibody or antibodies to one or more cell surface markers, for example, in immunolocalization, e.g., flow cytometry or FACS. Selection can be accomplished using antibodies in conjunction with magnetic beads. Antibodies that are specific for certain markers are known in the art and are available commercially, e.g., antibodies to CD9 (Abcam); CD54 (Abcam); CD105 (Abcam; BioDesign International, Saco, Me., etc.); CD200 (Abcam) cytokeratin (SigmaAldrich). Antibodies to other markers are also available commercially, e.g., CD34, CD38 and CD45 are available from, e.g., StemCell Technologies or BioDesign International. Primers to OCT-4 sequences suitable for RT-PCR can be obtained commercially, e.g., from Millipore or Invitrogen, or can be readily derived from the human sequence in GenBank Accession No. DQ486513.

Detailed methods of obtaining placenta and amnion tissue, and treating such tissue in order to obtain amnion derived adherent cells, are provided below.

4.5.1 Cell Collection Composition

Generally, cells can be obtained from amnion from a mammalian placenta, e.g., a human placenta, using a physiologically-acceptable solution, e.g., a cell collection composition. Preferably, the cell collection composition prevents or suppresses apoptosis, prevents or suppresses cell death, lysis, decomposition and the like. A cell collection composition is described in detail in related U.S. Patent Application Publication No. 2007/0190042, entitled “Improved Medium for Collecting Placental Stem Cells and Preserving Organs,” the disclosure of which is incorporated herein by reference in its entirety.

The cell collection composition can comprise any physiologically-acceptable solution suitable for the collection and/or culture of amnion derived adherent cells, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like, with or without the addition of a buffering component, e.g., 4-(2-hydroxyethyl)-1-piperazineethanesulfonic acid (HEPES).

The cell collection composition can comprise one or more components that tend to preserve cells, e.g., amnion derived adherent cells, that is, prevent the cells from dying, or delay the death of the cells, reduce the number of cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE™, hyaluronidase, and the like. The use of cell collection compositions comprising tissue-digesting enzymes is discussed in more detail below.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/1 to about 100 g/l, or about 40 g/1 to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

The amnion derived adherent cells described herein can also be collected, e.g., during and after digestion as described below, into a simple physiologically-acceptable buffer, e.g., phosphate-buffered saline, a 0.9% NaCl solution, cell culture medium, or the like.

4.5.2 Collection and Handling of Placenta

Generally, a human placenta is recovered shortly after its expulsion after birth, or after, e.g., Caesarian section. In a preferred embodiment, the placenta is recovered from a patient after informed consent and after a complete medical history of the patient is obtained and is associated with the placenta. Preferably, the medical history continues after delivery. Such a medical history can be used to coordinate subsequent use of the placenta or cells harvested therefrom. For example, human placental cells, e.g., amnion derived adherent cells, can be used, in light of the medical history, for personalized medicine for the infant, or a close relative, associated with the placenta, or for parents, siblings, or other relatives of the infant.

Prior to recovery of amnion derived adherent cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to cell collection, can be stored under sterile conditions and at a temperature of, e.g., 4 to 25° C. (centigrade), e.g., at room temperature. The placenta may be stored for, e.g., a period of for zero to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta can be stored in an anticoagulant solution at a temperature of, e.g., 4 to 25° C. (centigrade). Suitable anticoagulant solutions are well known in the art. For example, a solution of sodium citrate, heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before cells are collected.

4.5.3 Physical Disruption and Enzymatic Digestion of Amnion Tissue

In one embodiment, the amnion is separated from the rest of the placenta, e.g., by blunt dissection, e.g., using the fingers. The amnion can be dissected, e.g., into parts or tissue segments, prior to enzymatic digestion and adherent cell recovery. Amnion derived adherent cells can be obtained from a whole amnion, or from a small segment of amnion, e.g., a segment of amnion that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 square millimeters in area.

Amnion derived adherent cells can generally be collected from a placental amnion or a portion thereof, at any time within about the first three days post-expulsion, but preferably between about 0 hours and 48 hours after expulsion, or about 8 hours and about 18 hours post-expulsion.

In one embodiment, amnion derived adherent cells are extracted from amnion tissue by enzymatic digestion using one or more tissue-digesting enzymes. The amnion, or a portion thereof, may, e.g., be digested with one or more enzymes dissolved or mixed into a cell collection composition as described above.

In certain embodiments, the cell collection composition comprises one or more tissue-disruptive enzyme(s). Enzymatic digestion preferably uses a combination of enzymes, e.g., a combination of a matrix metalloprotease and a neutral protease, for example, a combination of dispase and collagenase, e.g., used in sequential order. When more than one protease is used, the proteases may be used at the same time to digest the amnion tissue, or may be used serially. In one embodiment, for example, the amnion tissue is digested three times with trypsin and then once with collagenase.

In one embodiment, amnion tissue is enzymatically digested with one or more of a matrix metalloprotease, a neutral protease, and a mucolytic enzyme. In a specific embodiment, the amnion tissue is digested with a combination of collagenase, dispase, and hyaluronidase. In another specific embodiment, the amnion tissue is digested with a combination of LIBERASE™ (Boehringer Mannheim Corp., Indianapolis, Ind.) and hyaluronidase. Other enzymes that can be used to disrupt amnion tissue include papain, deoxyribonucleases, serine proteases, such as trypsin, chymotrypsin, or elastase. Serine proteases may be inhibited by alpha 2 microglobulin in serum and therefore the medium used for digestion can, in certain embodiments, be serum-free. In certain other embodiments, EDTA and DNase are used in the digestion of amnion tissue, e.g., to increase the efficiency of cell recovery. In certain other embodiments, the digestate is diluted so as to avoid trapping cells within the viscous digest.

Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to isolate amnion derived adherent cells. For example, in one embodiment, amnion tissue, or part thereof, is digested first with an appropriate amount of trypsin, at a concentration of about 0.25%, for, e.g., 15 minutes, at 37° C., followed by collagenase I at about 1 to about 2 mg/ml for, e.g., 45 minutes.

In one embodiment, amnion derived adherent cells can be obtained as follows. The amniotic membrane is cut into segments approximately 0.1″×0.1″ to about 5″×5″, e.g., 2″×2″, in size. The epithelial monolayer is removed from the fetal side of the amniotic membrane by triple trypsinization as follows. The segments of amniotic membrane are placed into a container with warm (e.g., about 20° C. to about 37° C.) trypsin-EDTA solution (0.25%). The volume of trypsin can range from about 5 mL per gram of amnion to about 50 mL per gram of amnion. The container is agitated for about 5 minutes to about 30 minutes, e.g., 15 minutes, while maintaining the temperature constant. The segments of amniotic membrane are then separated from the trypsin solution by any appropriate method, such as manually removing the amnion segments, or by filtration. The trypsinization step can be repeated at least one more time.

Upon completion of the final trypsinization, the segments of amniotic membrane are placed back into the container filled with warm trypsin neutralization solution, such as phosphate-buffered saline (PBS)/10% FBS, PBS/5% FBS or PBS/3% FBS. The container is agitated for about 5 seconds to about 30 minutes, e.g., 5 minutes. The segments of amniotic membrane are then separated from the trypsin neutralization solution as described above, and the segments of amniotic membrane are placed into the container filled with warm PBS, pH 7.2. The container is agitated for about 5 seconds to about 30 minutes, and the amniotic membrane segments are then separated from the PBS as described above.

The segments of amniotic membrane are then placed into the container filled with warm (e.g., about 20° C. to about 37° C.) digestion solution. The volume of digestion solution can range from about 5 mL per gram of amnion to about 50 mL per gram of amnion. Digestion solutions comprise digestion enzymes in an appropriate culture medium, such as DMEM. Typical digestion solutions include collagenase type I (about 50 U/mL to about 500 U/mL); collagenase type I (about 50 U/mL to about 500 U/mL) plus dispase (about 5 U/mL to about 100 U/mL); and collagenase type I (about 50 U/mL to about 500 U/mL), dispase (about 2 U/mL to about 50 U/mL) and hyaluronidase (about 3 U/mL to about 10 U/mL). The container is agitated at 37° C. until amnion digestion is substantially complete (approximately 10 minutes to about 90 minutes). Warm PBS/5% FBS is then added to the container at a ratio of about 1 mL per gram of amniotic tissue to about 50 mL per gram of amniotic tissue. The container is agitated for about 2 minutes to about 5 minutes. The cell suspension is then filtered to remove any un-digested tissue using a 40 μm to 100 μm filter. The cells are suspended in warm PBS (about 1 mL to about 500 mL), and then centrifuged at 200×g to about 400×g for about 5 minutes to about 30 minutes, e.g. 300×g for about 15 minutes at 20° C. After centrifugation, the supernatant is removed and the cells are resuspended in a suitable culture medium. The cell suspension can be filtered (40 μm to 70 μm filter) to remove any remaining undigested tissue, yielding a single cell suspension.

In this embodiment, cells in suspension are collected and cultured as described elsewhere herein to produce isolated amnion derived adherent cells, and populations of such cells. The remaining undigested amnion, in this embodiment, can be discarded. The cells released from the amnion tissue can be, e.g., collected, e.g., by centrifugation, and cultured in standard cell culture medium.

In any of the digestion protocols herein, the cell suspension obtained by digestion can be filtered, e.g., through a filter comprising pores from about 50 μm to about 150 μm, e.g., from about 75 μm to about 125 μm. In a more specific embodiment, the cell suspension can be filtered through two or more filters, e.g., a 125 μm filter and a 75 μm filter.

In conjunction with any of the methods described herein, AMDACs can be isolated from the cells released during digestion by selecting cells that express one or more characteristics of AMDACs, as described in Section 4.1, above.

AMDACs can also, for example, be isolated using a specific two-step isolation method comprising digestion with trypsin followed by digestion with collagenase. Thus, in another aspect, provided herein is a method of isolating amnion derived adherent cells comprising digesting an amniotic membrane or portion thereof with trypsin such that epithelial cells are released from said amniotic membrane; removing the amniotic membrane or portion thereof from said epithelial cells; further digesting the amniotic membrane or portion thereof with collagenase such that amnion derived adherent cells are released from said amniotic membrane or portion thereof; and separating said amnion derived adherent cells from said amniotic membrane. In a specific embodiment, digestion of the amniotic membrane or portion thereof is repeated at least once. In another specific embodiment, digestion of the amniotic membrane or portion thereof with collagenase is repeated at least once. In another specific embodiment, the trypsin is at about 0.1%-1.0% (final concentration). In a more specific embodiment, the trypsin is at about 0.25% (final concentration). In another specific embodiment, the collagenase is at about 50 U/mL to about 1000 U/mL (final concentration). In a more specific embodiment, the collagenase is at about 125 U/mL (final concentration). In another specific embodiment, the method of isolation additionally comprises culturing said amnion derived adherent cells in cell culture and separating said amnion derived adherent cells from non-adherent cells in said culture to produce an enriched population of amnion derived adherent cells. In more specific embodiments, at least 50%, 55%, 60%, 65%, 70%, 75%, 80%, 85%, 90%, 95%, 98% or 99% of cells in said enriched population of amnion derived adherent cells are said amnion derived adherent cells.

In a more specific embodiment of the above methods, the amnion derived adherent cells are negative for OCT-4, as determined by RT-PCR, and one or more of HLA-G⁺, CD90⁺, CD105⁺, and CD117⁻, as determined by flow cytometry.

4.5.4 Isolation, Sorting, and Characterization of Amnion Derived Adherent Cells

Cell pellets can be resuspended in fresh cell collection composition, as described above, or a medium suitable for cell maintenance, e.g., Dulbecco's Modified Eagle's Medium (DMEM); Iscove's Modified Dulbecco's Medium (IMDM), e.g. IMDM serum-free medium containing 2U/mL heparin and 2 mM EDTA (GibcoBRL, NY); a mixture of buffer (e.g. PBS, HBSS) with FBS (e.g. 2% v/v); or the like.

Amnion derived adherent cells that have been cultured, e.g., on a surface, e.g., on tissue culture plastic, with or without additional extracellular matrix coating such as fibronectin, can be passaged or isolated by differential adherence. For example, a cell suspension obtained from collagenase digestion of amnion tissue, performed as described in Section 4.4.3, above, can be cultured, e.g., for 3-7 days in culture medium on tissue culture plastic. During culture, a plurality of cells in the suspension adhere to the culture surface, and, after continued culture, give rise to amnion derived adherent cells. Nonadherent cells, which do not give rise to the amnion derived adherent cells, are removed during medium exchange.

The number and type of cells collected from amnion can be monitored, for example, by measuring changes in morphology and cell surface markers using standard cell detection techniques such as immunolocalization, e.g., flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using one or more antibodies to CD34, one can determine, using the techniques above, whether a cell comprises a detectable amount of CD34; if so, the cell is CD34⁺.

Amnion-derived cells, e.g., cells that have been isolated by Ficoll separation, differential adherence, or a combination of both, can be sorted using a fluorescence activated cell sorter (FACS). Fluorescence activated cell sorting (FACS) is a well-known method for separating particles, including cells, based on the fluorescent properties of the particles (see, e.g., Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, cells from placenta, e.g., amnion derived adherent cells, can be sorted on the basis of expression of the markers CD49f, VEGFR2/KDR, and/or Flt-1/VEGFR1. Preferably the cells are identified as being OCT-4⁻, e.g., by determining the expression of OCT-4 by RT-PCR in a sample of the cells, wherein the cells are OCT-4⁻ if the cells in the sample fail to show detectable production of mRNA for OCT-4 after 30 cycles. For example, cells from amnion that are VEGFR2/KDR⁺ and VEGFR1/Flt-1⁺ can be sorted from cells that are one or more of VEGFR2/KDR⁻, and VEGFR1/Flt-1⁺, CD9⁺, CD54⁺, CD105⁺, CD200⁺, and/or VE-cadherin⁻. In a specific embodiment, amnion-derived, tissue culture plastic-adherent cells that are one or more of CD49f⁺, VEGFR2/KDR⁺, CD9⁺, CD54⁺, CD105⁺, CD200⁺, and/or VE-cadherin⁻, or cells that are VEGFR2/KDR⁺, CD9⁺, CD54⁺, CD105⁺, CD200⁺, and VE-cadherin⁻, are sorted away from cells not expressing one or more of such marker(s), and selected. In another specific embodiment, CD49f⁺, VEGFR2/KDR⁺, VEGFR1/Flt-1⁺ cells that are additionally one or more, or all, of CD31⁺, CD34⁺, CD45⁺, CD133⁻, and/or Tie-2⁺ are sorted from cells that do not display one or more, or any, of such characteristics. In another specific embodiment, VEGFR2/KDR⁺, VEGFR1/Flt-1⁺ cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and/or CXCR4⁻, are sorted from cells that do not display one or more, or any, of such characteristics.

Selection for amnion derived adherent cells can be performed on a cell suspension resulting from digestion, or on isolated cells collected from digestate, e.g., by centrifugation or separation using flow cytometry. Selection by expressed markers can be accomplished alone or, e.g., in connection with procedures to select cells on the basis of their adherence properties in culture. For example, an adherence selection can be accomplished before or after sorting on the basis of marker expression.

With respect to antibody-mediated detection and sorting of placental cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against CD105 (available from R&D Systems Inc., Minneapolis, Minn.); phycoerythrin (PE) conjugated monoclonal antibodies against CD200 (BD Biosciences Pharmingen); VEGFR2/KDR-Biotin (CD309, Abcam), and the like. Antibodies to any of the markers disclosed herein can be labeled with any standard label for antibodies that facilitates detection of the antibodies, including, e.g., horseradish peroxidase, alkaline phosphatase, β-galactosidase, acetylcholinesterase streptavidin/biotin, avidin/biotin, umbelliferone, fluorescein, fluorescein isothiocyanate (FITC), rhodamine, dichlorotriazinylamine fluorescein, dansyl chloride or phycoerythrin (PE), luminol, luciferase, luciferin, and aequorin, and examples of suitable radioactive material include ¹²⁵I, ¹³¹I, ³⁵S or ³H.

Amnion derived adherent cells can be labeled with an antibody to a single marker and detected and/sorted based on the single marker, or can be simultaneously labeled with multiple antibodies to a plurality of different markers and sorted based on the plurality of markers.

In another embodiment, magnetic beads can be used to separate cells, e.g., to separate the amnion derived adherent cells described herein from other amnion cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Amnion derived adherent cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay or MTT cell proliferation assay (to assess proliferation). Longevity may be determined by methods well known in the art, such as by determining the maximum number of population doubling in an extended culture.

Amnion derived adherent cells, can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

4.6 Culture of Amnion Derived Adherent Cells

4.6.1 Culture Media

Isolated amnion derived adherent cells, or populations of such cells, can be used to initiate, or seed, cell cultures. Cells are generally transferred to sterile tissue culture vessels either uncoated or coated with extracellular matrix or biomolecules such as laminin, collagen (e.g., native or denatured), gelatin, fibronectin, ornithine, vitronectin, and extracellular membrane protein (e.g., MATRIGEL™ (BD Discovery Labware, Bedford, Mass.)).

AMDACs can, for example, be established in media suitable for the culture of stem cells, Establishment media can, for example, include EGM-2 medium (Lonza), DMEM+10% FBS, or medium comprising 60% DMEM-LG (Gibco), 40% MCDB-201(Sigma), 2% fetal calf serum (FCS) (Hyclone Laboratories), 1× insulin-transferrin-selenium (ITS), 1× lenolenic-acid-bovine-serum-albumin (LA-BSA), 10⁻⁹ M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin (referred to herein as “standard medium”).

Amnion derived adherent cells can be cultured in any medium, and under any conditions, recognized in the art as acceptable for the culture of cells, e.g., adherent placental stem cells. Preferably, the culture medium comprises serum. In various embodiments, media for the culture or subculture of AMDACs includes STEMPRO® (Invitrogen), MSCM-sf (ScienCell, Carlsbad, Calif.), MESENCULT®-ACF medium (StemCell Technologies, Vancouver, Canada), standard medium, standard medium lacking EGF, standard medium lacking PDGF, DMEM+10% FBS, EGM-2 (Lonza), EGM-2MV (Lonza), 2%, 10% and 20% ES media, ES-SSR medium, or α-MEM-20% FBS. Medium acceptable for the culture of amnion derived adherent cells includes, e.g., DMEM, IMDM, DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM Lonza), ADVANCESTEM™ Medium (Hyclone), KNOCKOUT™ DMEM (Invitrogen), Leibovitz's L-15 medium, MCDB, DMEM/F12, RPMI 1640, advanced DMEM (Gibco), DMEM/MCDB201 (Sigma), and CELL-GRO FREE, or the like. In various embodiments, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising about 2 to about 20%, e.g., about 10%, fetal bovine serum (FBS; e.g. defined fetal bovine serum, Hyclone, Logan Utah); DMEM-HG comprising about 2 to about 20%, e.g., about 15%, FBS; IMDM (Iscove's modified Dulbecco's medium) comprising about 2 to about 20%, e.g., about 10%, FBS, about 2 to about 20%, e.g., about 10%, horse serum, and hydrocortisone; M199 comprising about 2 to about 20%, e.g., about 10%, FBS, EGF, and heparin; α-MEM (minimal essential medium) comprising about 2 to about 20%, e.g., about 10%, FBS, GLUTAMAX™ and gentamicin; DMEM comprising 10% FBS, GLUTAMAX™ and gentamicin; DMEM-LG comprising about 2 to about 20%, e.g., about 15%, (v/v) fetal bovine serum (e.g., defined fetal bovine serum, Hyclone, Logan Utah), antibiotics/antimycotics (e.g., penicillin at about 100 Units/milliliter, streptomycin at 100 micrograms/milliliter, and/or amphotericin B at 0.25 micrograms/milliliter (Invitrogen, Carlsbad, Calif.)), and 0.001% (v/v) β-mercaptoethanol (Sigma, St. Louis Mo.); KNOCKOUT™-DMEM basal medium supplemented with 2 to 20% FBS, non-essential amino acid (Invitrogen), beta-mercaptoethanol, KNOCKOUT™ basal medium supplemented with KNOCKOUT™ Serum Replacement, alpha-MEM comprising 2 to 20% FBS, EBM2™ basal medium supplemented with EGF, VEGF, bFGF, R3-IGF-1, hydrocortisone, heparin, ascorbic acid, FBS, gentamicin), or the like.

The culture medium can be supplemented with one or more components including, for example, serum (e.g., FCS or FBS, e.g., about 2-20% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

Amnion derived adherent cells (AMDACs) can be cultured in standard tissue culture conditions, e.g., in tissue culture dishes or multiwell plates. The cells can also be cultured using a hanging drop method. In this method, the cells are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the cells are cultured. AMDACs can also be cultured in standard or high-volume or high-throughput culture systems, such asT-flasks, Corning HYPERFLASK®, Cell Factories (Nunc), 1-, 2-, 4-, 10 or 40-Tray Cell stacks, and the like.

In one embodiment, amnion derived adherent cells are cultured in the presence of a compound that acts to maintain an undifferentiated phenotype in the cells. In a specific embodiment, the compound is a substituted 3,4-dihydropyridimol[4,5-d]pyrimidine. In a more specific embodiment, the compound is a compound having the following chemical structure:

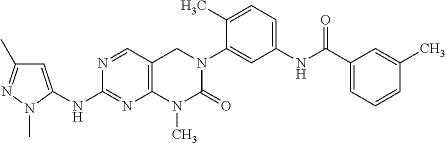

The compound can be contacted with an amnion derived adherent cell, or population of such cells, at a concentration of, for example, between about 1 μM to about 10 μM.

4.6.2 Expansion and Proliferation of Amnion Derived Adherent Cells

Once an isolated amnion derived adherent cell, or isolated population of such cells (e.g., amnion derived adherent cells, or population of such cells separated from at least 50% of the amnion cells with which the cell or population of cells is normally associated in vivo), the cells can be proliferated and expanded in vitro. For example, a population of adherent cells or amnion derived adherent cells can be cultured in tissue culture containers, e.g., dishes, flasks, multiwell plates, or the like, for a sufficient time for the cells to proliferate to 40-70% confluence, that is, until the cells and their progeny occupy 40-70% of the culturing surface area of the tissue culture container.

Amnion derived adherent cells can be seeded in culture vessels at a density that allows cell growth. For example, the cells may be seeded at low density (e.g., about 400 to about 6,000 cells/cm²) to high density (e.g., about 20,000 or more cells/cm²). In a preferred embodiment, the cells are cultured at about 0% to about 5% by volume CO₂ in air. In some preferred embodiments, the cells are cultured at about 0.1% to about 25% O₂ in air, preferably about 5% to about 20% O₂ in air. The cells are preferably cultured at about 25° C. to about 40° C., preferably at about 37° C.

The cells are preferably cultured in an incubator. During culture, the culture medium can be static or can be agitated, for example, during culture using a bioreactor. Amnion derived adherent cells preferably are grown under low oxidative stress (e.g., with addition of glutathione, ascorbic acid, catalase, tocopherol, N-acetylcysteine, or the like).

Although the amnion-derived angiogenic cells may be grown to confluence, the cells are preferably not grown to confluence. For example, once 40%-70% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 cells, preferably about 50,000 cells, or about 400 to about 6,000 cells/cm², can be passaged to a new culture container containing fresh culture medium. Typically, the new medium is the same type of medium from which the cells were removed. The amnion derived adherent cells can be passaged at least 1, 2, 3, 4, 5, 6, 7, 8, 9, 10, 11, 12, 13, 14, 15, 16, 17, 18, 19 or 20 times, or more. AMDACs can be doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

4.7 Preservation of Amnion Derived Adherent Cells

Amnion derived adherent cells can be preserved, that is, placed under conditions that allow for long-term storage, or conditions that inhibit cell death by, e.g., apoptosis or necrosis, e.g., during collection or prior to production of the compositions described herein, e.g., using the methods described herein.

Amnion derived adherent cells can be preserved using, e.g., a composition comprising an apoptosis inhibitor, necrosis inhibitor and/or an oxygen-carrying perfluorocarbon, as described in U.S. Application Publication No. 2007/0190042, the disclosure of which is hereby incorporated by reference in its entirety. In one embodiment, a method of preserving such cells, or a population of such cells, comprises contacting said cells or population of cells with a cell collection composition comprising an inhibitor of apoptosis and an oxygen-carrying perfluorocarbon, wherein said inhibitor of apoptosis is present in an amount and for a time sufficient to reduce or prevent apoptosis in the population of cells, as compared to a population of cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of amnion derived adherent cells. In another embodiment, said cell collection composition comprises said inhibitor of apoptosis and said oxygen-carrying perfluorocarbon in separate phases. In another embodiment, said cell collection composition comprises said inhibitor of apoptosis and said oxygen-carrying perfluorocarbon in an emulsion. In another embodiment, the cell collection composition additionally comprises an emulsifier, e.g., lecithin. In another embodiment, said apoptosis inhibitor and said perfluorocarbon are between about 0° C. and about 25° C. at the time of contacting the cells. In another more specific embodiment, said apoptosis inhibitor and said perfluorocarbon are between about 2° C. and 10° C., or between about 2° C. and about 5° C., at the time of contacting the cells. In another more specific embodiment, said contacting is performed during transport of said population of cells. In another more specific embodiment, said contacting is performed during freezing and thawing of said population of cells.

Populations of amnion derived adherent cells can be preserved, e.g., by a method comprising contacting a population of said cells with an inhibitor of apoptosis and an organ-preserving compound, wherein said inhibitor of apoptosis is present in an amount and for a time sufficient to reduce or prevent apoptosis in the population of cells, as compared to a population of cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267. In another embodiment, said organ-preserving compound is hydroxyethyl starch, lactobionic acid, raffinose, or a combination thereof. In another embodiment, the cell collection composition additionally comprises an oxygen-carrying perfluorocarbon, either in two phases or as an emulsion.

In another embodiment of the method, amnion derived adherent cells are contacted with a cell collection composition comprising an apoptosis inhibitor and oxygen-carrying perfluorocarbon, organ-preserving compound, or combination thereof, during perfusion. In another embodiment, the amnion derived adherent cells are contacted with such a cell collection composition during a process of tissue disruption, e.g., enzymatic digestion of amnion tissue. In another embodiment, amnion derived adherent cells are contacted with said cell collection compound after collection by tissue disruption, e.g., enzymatic digestion of amnion tissue.

Typically, during collection of amnion derived adherent cells, enrichment and isolation, it is preferable to minimize or eliminate cell stress due to hypoxia and mechanical stress. In another embodiment of the method, therefore, an amnion derived adherent cell, or population of cells comprising the amnion derived adherent cells, is exposed to a hypoxic condition during collection, enrichment or isolation for less than six hours during said preservation, wherein a hypoxic condition is a concentration of oxygen that is, e.g., less than normal atmospheric oxygen concentration; less than normal blood oxygen concentration; or the like. In a more specific embodiment, said cells or population of said cells is exposed to said hypoxic condition for less than two hours during said preservation. In another more specific embodiment, said cells or population of said cells is exposed to said hypoxic condition for less than one hour, or less than thirty minutes, or is not exposed to a hypoxic condition, during collection, enrichment or isolation. In another specific embodiment, said population of cells is not exposed to shear stress during collection, enrichment or isolation.

Amnion derived adherent cells can be cryopreserved, in general or by the specific methods disclosed herein, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., cell freezing medium identified by SigmaAldrich catalog numbers C2695, C2639 (Cell Freezing Medium-Serum-free 1×, not containing DMSO) or C6039 (Cell Freezing Medium-Glycoerol 1× containing Minimum Essential Medium, glycerol, calf serum and bovine serum), Lonza PROFREEZE™ 2× Medium, methylcellulose, dextran, human serum albumin, fetal bovine serum, fetal calf serum, or Plasmalyte. Cryopreservation medium preferably comprises DMSO (dimethylsulfoxide) or glycerol, at a concentration of, e.g., about 1% to about 20%, e.g., about 5% to 10% (v/v), optionally including fetal bovine serum or human serum. Cryopreservation medium may comprise additional agents, for example, methylcellulose and/or glycerol. Isolated amnion derived adherent cells are preferably cooled at about 1° C./min during cryopreservation. A preferred cryopreservation temperature is about −80° C. to about −180° C., preferably about −125° C. to about −140° C. Cryopreserved cells can be transferred to vapor phase of liquid nitrogen prior to thawing for use. In some embodiments, for example, once the ampoules have reached about −80° C., they are transferred to a liquid nitrogen storage area. Cryopreservation can also be done using a controlled-rate freezer. Cryopreserved cells preferably are thawed at a temperature of about 25° C. to about 40° C., preferably to a temperature of about 37° C.

4.8 Production of a Bank of Amnion Derived Adherent Cells

Amnion derived adherent cells can be cultured in a number of different ways to produce a set of lots, e.g., a set of individually-administrable doses, of such cells. Sets of lots of angiogenic amniotic cells, obtained from a plurality of placentas, can be arranged in a bank of cells for, e.g., long-term storage. Generally, amnion derived adherent cells are obtained from an initial culture of cells to form a seed culture, which is expanded under controlled conditions to form populations of cells from approximately equivalent numbers of doublings. Lots are preferably derived from the tissue of a single placenta, but can be derived from the tissue of a plurality of placentas.

In one non-limiting embodiment, lots or doses of amnion derived adherent cells are obtained as follows. Amnion tissue is first disrupted, e.g., digested as described in Section 4.4.3, above using serial trypsin and collagenase digestions. Cells from the collagenase-digested tissue are cultured, e.g., for about 1-3 weeks, preferably about 2 weeks. After removal of non-adherent cells, high-density colonies that form are collected, e.g., by trypsinization. These cells are collected and resuspended in a convenient volume of culture medium, and defined as Passage 0 cells.

Passage 0 cells can then be used to seed expansion cultures. Expansion cultures can be any arrangement of separate cell culture apparatuses, e.g., a Cell Factory by NUNC™ Cells in the Passage 0 culture can be subdivided to any degree so as to seed expansion cultures with, e.g., 1×10³, 2×10³, 3×10³, 4×10³, 5×10³, 6×10³, 7×10³, 8×10³, 9×10³, 1×10⁴, 1×10⁴, 2×10⁴, 3×10⁴, 4×10⁴, 5×10⁴, 6×10⁴, 7×10⁴, 8×10⁴, 9×10⁴, or 10×10⁴ adherent cells. Preferably, from about 1×10³ to about 3×10⁴ Passage 0 cells are used to seed each expansion culture. The number of expansion cultures can depend upon the number of Passage 0 cells, and may be greater or fewer in number depending upon the particular placenta(s) from which the adherent cells are obtained.

Expansion cultures can then be grown until the density of cells in culture reaches a certain value, e.g., about 1×10⁵ cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 15, 16, 17, 18, 19 or 20 times prior to use. A record of the cumulative number of population doublings is preferably maintained during expansion culture(s). The cells from a Passage 0 culture can be expanded for 2, 3, 4, 5, 6, 7, 8, 9, 10, 12, 14, 16, 18, 20, 22, 24, 26, 28, 30, 32, 34, 36, 38 or 40 doublings, or up to 60 doublings. Preferably, however, the number of population doublings, prior to dividing the population of cells into individual doses, is between about 15 and about 30 doublings. The cells can be culture continuously throughout the expansion process, or can be frozen at one or more points during expansion.

Cells to be used for individual doses can be frozen, e.g., cryopreserved for later use. Individual doses can comprise, e.g., about 1 million to about 50 million cells per mL, and can comprise between about 10⁶ and about 10¹⁰ cells in total.

In one embodiment, therefore, a cell bank comprising amnion derived adherent cells can be made by a method comprising: expanding primary culture amnion derived adherent cells from a human post-partum placenta for a first plurality of population doublings; cryopreserving the cells to form a Master Cell Bank; optionally expanding a plurality of the cells from the Master Cell Bank for a second plurality of population doublings; cryopreserving the expanded cells to form a Working Cell Bank; optionally expanding a plurality of the expanded amnion derived adherent cells from the Working Cell Bank for a third plurality of population doublings; and cryopreserving the resulting expanded cells in individual doses, wherein said individual doses collectively compose a cell bank. The bank can comprise doses, or lots, of solely amnion derived adherent cells, or can comprise a combination of lots of amnion derived adherent cells and lots or doses of another kind of cell, e.g., another kind of stem or progenitor cell. Preferably, each individual dose comprises only amnion derived adherent cells. In another specific embodiment, all of said cells in said primary culture are from the same placenta. In another specific embodiment, said individual doses comprise from about 10⁴ to about 10⁵ cells. In another specific embodiment, said individual doses comprise from about 10⁵ to about 10⁶ cells. In another specific embodiment, said individual doses comprise from about 10⁶ to about 10⁷ cells. In another specific embodiment, said individual doses comprise from about 10⁷ to about 10⁸ cells. In another specific embodiment, said individual doses comprise from about 10⁸ to about 10⁹ cells. In another specific embodiment, said individual doses comprise from about 10⁹ to about 10¹⁰ cells.

In certain embodiments, amnion derived adherent cells can be thawed from a Working Cell Bank and cultured for a plurality of population doublings. When a desired number of cells is generated, or a desired number of population doublings has taken place, the adherent cells can be collected, e.g., by centrifugation, and resuspended in a solution comprising, e.g., dextran, e.g., 5% dextran. In certain embodiments, the dextran is dextran-40. In certain embodiments, the cells are collected a second time and resuspended in a solution comprising dextran and a cryopreservant, e.g., a 5% dextran (e.g., dextran-40) solution comprising 10% HSA and 2%-20%, e.g., 5% DMSO, and cryopreserved. The cryopreserved amnion derived adherent cells can be thawed, e.g., immediately before use.

In a preferred embodiment, the donor from which the placenta is obtained (e.g., the mother) is tested for at least one pathogen. In certain embodiments, if the mother tests positive for a tested pathogen, the entire lot from the placenta is discarded. Such testing can be performed at any time during production of lots of amnion derived adherent cells, including before or after establishment of Passage 0 cells, or during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

4.9 Uses of Amnion Derived Adherent Cells

Provided herein are compositions comprising amnion derived adherent cells. Examples of such compositions include pharmaceutical compositions (see Section 4.8.1, below); matrices and scaffolds (see Section 4.8.2, below), and media conditioned by amnion derived adherent cells (see Section 4.8.3, below).

4.9.1 Compositions Comprising Amnion Derived Adherent Cells

In certain embodiments, amnion derived adherent cells are contained within, or are components of, a pharmaceutical composition. The cells can be prepared in a form that is easily administrable to an individual, e.g., amnion derived adherent cells that are contained within a container that is suitable for medical use. Such a container can be, for example, a syringe, sterile plastic bag, flask, jar, or other container from which the anion derived angiogenic cell population can be easily dispensed. For example, the container can be a blood bag or other plastic, medically-acceptable bag suitable for the intravenous administration of a liquid to a recipient. The container, in certain embodiments, is one that allows for cryopreservation of the cells. The cells in the compositions, e.g., pharmaceutical compositions, provided herein, can comprise amnion derived adherent cells derived from a single donor, or from multiple donors. The cells can be completely HLA-matched to an intended recipient, or partially or completely HLA-mismatched.

Thus, in one embodiment, amnion derived adherent cells in the compositions provided herein are administered to an individual in need thereof in the form of a composition comprising amnion derived adherent cells in a container. In another specific embodiment, the container is a bag, flask, or jar. In more specific embodiment, said bag is a sterile plastic bag. In a more specific embodiment, said bag is suitable for, allows or facilitates intravenous administration of said adherent cells, e.g., by intravenous infusion, bolus injection, or the like. The bag can comprise multiple lumens or compartments that are interconnected to allow mixing of the cells and one or more other solutions, e.g., a drug, prior to, or during, administration. In another specific embodiment, prior to cryopreservation, the solution comprising the amnion derived adherent cells comprises one or more compounds that facilitate cryopreservation of the cells. In another specific embodiment, said amnion derived adherent cells are contained within a physiologically-acceptable aqueous solution. In a more specific embodiment, said physiologically-acceptable aqueous solution is a 0.9% NaCl solution. In another specific embodiment, said amnion derived adherent cells comprise placental cells that are HLA-matched to a recipient of said cells. In another specific embodiment, said amnion derived adherent cells comprise cells that are at least partially HLA-mismatched to a recipient of said cells. In another specific embodiment, said amnion derived adherent cells are derived from a plurality of donors. In various specific embodiments, said container comprises about, at least, or at most 1×10⁶ said cells, 5×10⁶ said cells, 1×10⁷ said stem cells, 5×10⁷ said cells, 1×10⁸ said cells, 5×10⁸ said cells, 1×10⁹ said cells, 5×10⁹ said cells, or 1×10¹⁰ said cells. In other specific embodiments of any of the foregoing cryopreserved populations, said cells have been passaged about, at least, or no more than 5 times, no more than 10 times, no more than 15 times, or no more than 20 times. In another specific embodiment of any of the foregoing cryopreserved cells, said cells have been expanded within said container. In specific embodiments, a single unit dose of amnion derived adherent cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more amnion derived adherent cells.

In certain embodiments, the pharmaceutical compositions provided herein comprises populations of amnion derived adherent cells, that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

4.9.2 Matrices Comprising Amnion Derived Adherent Cells

Further provided herein are compositions comprising matrices, hydrogels, scaffolds, and the like. Such compositions can be used in the place of, or in addition to, such cells in liquid suspension.

The matrix can be, e.g., a permanent or degradable decellularized tissue, e.g., a decellularized amniotic membrane, or a synthetic matrix. The matrix can be a three-dimensional scaffold. In a more specific embodiment, said matrix comprises collagen, gelatin, laminin, fibronectin, pectin, ornithine, or vitronectin. In another more specific embodiment, the matrix is an amniotic membrane or an amniotic membrane-derived biomaterial. In another more specific embodiment, said matrix comprises an extracellular membrane protein. In another more specific embodiment, said matrix comprises a synthetic compound. In another more specific embodiment, said matrix comprises a bioactive compound. In another more specific embodiment, said bioactive compound is a growth factor, a cytokine, an antibody, or an organic molecule of less than 5,000 daltons.

The amnion derived adherent cells described herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which the amnion derived adherent cells provided herein can be seeded are described in Hariri, U.S. Application Publication No. 2004/0048796, the disclosure of which is incorporated by reference herein in its entirety.

In another specific embodiment, the matrix is a composition comprising an extracellular matrix. In a more specific embodiment, said composition is MATRIGEL™ (BD Biosciences).

The isolated amnion derived adherent cells described herein can be suspended in a hydrogel solution suitable for, e.g., injection. The hydrogel is, e.g., an organic polymer (natural or synthetic) that is cross-linked via covalent, ionic, or hydrogen bonds to create a three-dimensional open-lattice structure that entraps water molecules to form a gel. Suitable hydrogels for such compositions include self-assembling peptides, such as RAD 16. In one embodiment, a hydrogel solution comprising the cells can be allowed to harden, for instance in a mold, to form a matrix having cells dispersed therein for implantation. The amnion derived adherent cells in such a matrix can also be cultured so that the cells are mitotically expanded, e.g., prior to implantation. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In certain embodiments, the compositions comprising cells, provided herein, comprise an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003). In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

In a specific embodiment, the matrix is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another preferred embodiment the cells of the invention are seeded onto foam scaffolds that may be composite structures. In addition, the three-dimensional framework may be molded into a useful shape, such as a specific structure in the body to be repaired, replaced, or augmented. Other examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

The amnion derived adherent cells described herein can be seeded onto a three-dimensional framework or scaffold and implanted in vivo. Such a framework can be implanted in combination with any one or more growth factors, cells, drugs or other components that, e.g., stimulate tissue formation, e.g., bone formation or formation of vasculature.

The placental amnion derived adherent cells provided herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the matrix comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The matrix can also comprise anti-thrombotic agents such as heparin; the scaffolds can also be treated to alter the surface charge (e.g., coating with plasma) prior to seeding with the adherent cells provided herein.

The framework may be treated prior to inoculation of the amnion derived adherent cells provided herein in order to enhance cell attachment. For example, prior to inoculation with the cells of the invention, nylon matrices could be treated with 0.1 molar acetic acid and incubated in polylysine, PBS, and/or collagen to coat the nylon. Polystyrene can be similarly treated using sulfuric acid.

In addition, the external surfaces of the three-dimensional framework may be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, or plant gums.

In some embodiments, the matrix comprises or is treated with materials that render the matrix non-thrombogenic, e.g., natural materials such as basement membrane proteins such as laminin and Type IV collagen, and synthetic materials such as ePTFE or segmented polyurethaneurea silicones, such as PURSPAN (The Polymer Technology Group, Inc., Berkeley, Calif.). Such materials can be further treated to render the scaffold non-thrombogenic, e.g., with heparin, and treatments that alter the surface charge of the material, such as plasma coating.

The therapeutic cell compositions comprising amnion derived adherent cells can also be provided in the form of a matrix-cell complex. Matrices can include biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. In certain embodiments, the cells adhere to the matrix. In other embodiments, the cells are entrapped or contained within matrix spaces. Most preferred are those matrix-cell complexes in which the cells grow in close association with the matrix and when used therapeutically, stimulate and support ingrowth of a recipient's cells, or stimulate or support angiogenesis. The matrix-cell compositions can be introduced into an individual's body in any way known in the art, including but not limited to implantation, injection, surgical attachment, transplantation with other tissue, injection, and the like. In some embodiments, the matrices form in vivo, or in situ. For example, in situ polymerizable gels can be used in accordance with the invention. Examples of such gels are known in the art.

In some embodiments, the cells provided herein are seeded onto such three-dimensional matrices, such as scaffolds and implanted in vivo, where the seeded cells may proliferate on or in the framework or help establish replacement tissue in vivo with or without cooperation of other cells. Growth of the amnion derived adherent cells or co-cultures thereof on the three-dimensional framework preferably results in the formation of a three-dimensional tissue, or foundation thereof, which can be utilized in vivo, for example for repair of damaged or diseased tissue. For example, the three-dimensional scaffolds can be used to form tubular structures, for example for use in repair of blood vessels; or aspects of the circulatory system or coronary structures. In accordance with one aspect of the invention, amnion derived adherent cells, or co-cultures thereof, are inoculated, or seeded on a three-dimensional framework or matrix, such as a scaffold, a foam or hydrogel. The framework may be configured into various shapes such as generally flat, generally cylindrical or tubular, or can be completely free-form as may be required or desired for the corrective structure under consideration. In some embodiments, the amnion derived adherent cells grow on the three dimensional structure, while in other embodiments, the cells only survive, or even die, but stimulate or promote ingrowth of new tissue or vascularization in a recipient.

The cells of the invention can be grown freely in culture, removed from the culture and inoculated onto a three-dimensional framework. Inoculation of the three-dimensional framework with a concentration of cells, e.g., approximately 10⁶ to 5×10⁷ cells per milliliter, preferably results in the establishment of the three-dimensional support in relatively shorter periods of time. Moreover in some application it may be preferably to use a greater or lesser number of cells depending on the result desired.

In a specific embodiment, the matrix can be cut into a strip (e.g., rectangular in shape) of which the width is approximately equal to the inner circumference of a tubular organ into which it will ultimately be inserted. The amnion derived adherent cells can be inoculated onto the scaffold and incubated by floating or suspending in liquid media. At the appropriate stage of confluence, the scaffold can be rolled up into a tube by joining the long edges together. The seam can then be closed by suturing the two edges together using fibers of a suitable material of an appropriate diameter. In order to prevent cells from occluding the lumen, one of the open ends of the tubular framework can be affixed to a nozzle. Liquid media can be forced through the nozzle from a source chamber connected to the incubation chamber to create a current through the interior of the tubular framework. The other open end can be affixed to an outflow aperture which leads into a collection chamber from which the media can be recirculated through the source chamber. The tube can be detached from the nozzle and outflow aperture when incubation is complete. See, e.g., International Application No. WO 94/25584.

In general, two three-dimensional frameworks can be combined into a tube in accordance with the invention using any of the following methods. Two or more flat frameworks can be laid atop another and sutured together. The resulting two-layer sheet can then be rolled up, and, as described above, joined together and secured. In certain embodiments, one tubular scaffold that is to serve as the inner layer can be inoculated with amnion derived adherent cells and incubated. A second scaffold can be grown as a flat strip with width slightly larger than the outer circumference of the tubular framework. After appropriate growth is attained, the flat framework is wrapped around the outside of the tubular scaffold followed by closure of the seam of the two edges of the flat framework and securing the flat framework to the inner tube. In another embodiment, two or more tubular meshes of slightly differing diameters can be grown separately. The framework with the smaller diameter can be inserted inside the larger one and secured. For each of these methods, more layers can be added by reapplying the method to the double-layered tube. The scaffolds can be combined at any stage of growth of the amnion derived adherent cells, and incubation of the combined scaffolds can be continued when desirable.

In conjunction with the above, the cells and therapeutic compositions provided herein can be used in conjunction with implantable devices. For example the amnion derived adherent cells can be coadminstered with, for example, stents, artificial valves, ventricular assist devices, Guglielmi detachable coils and the like. As the devices may constitute the dominant therapy provided to an individual in need of such therapy, the cells and the like may be used as supportive or secondary therapy to assist in, stimulate, or promote proper healing in the area of the implanted device. The cells and therapeutic compositions of the invention may also be used to pretreat certain implantable devices, to minimize problems when they are used in vivo. Such pretreated devices, including coated devices, may be better tolerated by patients receiving them, with decrease risk of local or systemic infection, or for example, restenosis or further occlusion of blood vessels.

4.9.3 Media Conditioned by Amnion Derived Adherent Cells

Further provided herein is medium that has been conditioned by amnion derived adherent cells, that is, medium comprising one or more biomolecules secreted or excreted by the adherent cells. In various embodiments, the conditioned medium comprises medium in which the cells have grown for at least 1, 2, 3, 4, 5, 6, 7, 8, 9, 10, 11, 12, 13, 14 or more days, or for at least 1, 2, 3, 4, 5, 6, 7, 8, 9, 10, 11, 12, 13, 14, 15, 16, 17, 18, 19, or 20 population doublings, or more. In other embodiments, the conditioned medium comprises medium in which amnion derived adherent cells have grown to at least 30%, 40%, 50%, 60%, 70%, 80%, 90% confluence, or up to 100% confluence. Such conditioned medium can be used to support the culture of a population of cells, e.g., stem cells, e.g., placental stem cells, embryonic stem cells, embryonic germ cells, adult stem cells, or the like. In another embodiment, the conditioned medium comprises medium in which amnion derived adherent cells, and cells that are not amnion derived adherent cells, have been cultured together.

The conditioned medium can comprise the adherent cells provided herein. Thus, provided herein is a cell culture comprising amnion derived adherent cells. In a specific embodiment, the conditioned medium comprises a plurality, e.g., a population, of amnion derived adherent cells.

4.10 Modified Amnion Derived Adherent Cells

4.10.1 Genetically Modified Amnion Derived Adherent Cells

In another aspect, the amnion derived adherent cells described herein can be genetically modified, e.g., to produce a nucleic acid or polypeptide of interest, or to produce a differentiated cell, e.g., an osteogenic cell, myocytic cell, pericytic cell, or angiogenic cell, that produces a nucleic acid or polypeptide of interest. For example, the amnion derived adherent cells can be modified to produce angiogenic factors, such as proangiogenic molecules, soluble factors and receptors or promigratory molecules such as chemokines, e.g., stromal cell derived factor 1 (SDF-1) or chemokine receptors. Genetic modification can be accomplished, e.g., using virus-based vectors including, but not limited to, non-integrating replicating vectors, e.g., papilloma virus vectors, SV40 vectors, adenoviral vectors; integrating viral vectors, e.g., retrovirus vector or adeno-associated viral vectors; or replication-defective viral vectors. Other methods of introducing DNA into cells include the use of liposomes, electroporation, a particle gun, direct DNA injection, or the like.

The adherent cells provided herein can be, e.g., transformed or transfected with DNA controlled by or in operative association with, one or more appropriate expression control elements, for example, promoter or enhancer sequences, transcription terminators, polyadenylation sites, internal ribosomal entry sites. Preferably, such a DNA incorporates a selectable marker. Following the introduction of the foreign DNA, engineered adherent cells can be, e.g., grown in enriched media and then switched to selective media. In one embodiment, the DNA used to engineer a amnion derived adherent cell comprises a nucleotide sequence encoding a polypeptide of interest, e.g., a cytokine, growth factor, differentiation agent, or therapeutic polypeptide.

The DNA used to engineer the adherent cell can comprise any promoter known in the art to drive expression of a nucleotide sequence in mammalian cells, e.g., human cells. For example, promoters include, but are not limited to, CMV promoter/enhancer, SV40 promoter, papillomavirus promoter, Epstein-Barr virus promoter, elastin gene promoter, and the like. In a specific embodiment, the promoter is regulatable so that the nucleotide sequence is expressed only when desired. Promoters can be either inducible (e.g., those associated with metallothionein and heat shock proteins) or constitutive.

In another specific embodiment, the promoter is tissue-specific or exhibits tissue specificity. Examples of such promoters include but are not limited to myosin light chain-2 gene control region (Shani, 1985, Nature 314:283) (skeletal muscle).

The amnion derived adherent cells disclosed herein may be engineered or otherwise selected to “knock out” or “knock down” expression of one or more genes in such cells. The expression of a gene native to a cell can be diminished by, for example, inhibition of expression by inactivating the gene completely by, e.g., homologous recombination. In one embodiment, for example, an exon encoding an important region of the protein, or an exon 5′ to that region, is interrupted by a positive selectable marker, e.g., neo, preventing the production of normal mRNA from the target gene and resulting in inactivation of the gene. A gene may also be inactivated by creating a deletion in part of a gene or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084). Antisense, morpholinos, DNAzymes, small interfering RNA, short hairpin RNA, and ribozyme molecules that inhibit expression of the target gene can also be used to reduce the level of target gene activity in the adherent cells. For example, antisense RNA molecules which inhibit the expression of major histocompatibility gene complexes (HLA) have been shown to be most versatile with respect to immune responses. Triple helix molecules can be utilized in reducing the level of target gene activity. See, e.g., L. G. Davis et al. (eds), 1994, BASIC METHODS IN MOLECULAR BIOLOGY, 2nd ed., Appleton & Lange, Norwalk, Conn., which is incorporated herein by reference.

In a specific embodiment, the amnion derived adherent cells disclosed herein can be genetically modified with a nucleic acid molecule comprising a nucleotide sequence encoding a polypeptide of interest, wherein expression of the polypeptide of interest is controllable by an exogenous factor, e.g., polypeptide, small organic molecule, or the like. The polypeptide of interest can be a therapeutic polypeptide. In a more specific embodiment, the polypeptide of interest is IL-12 or interleukin-1 receptor antagonist (IL-1Ra). In another more specific embodiment, the polypeptide of interest is a fusion of interleukin-1 receptor antagonist and dihydrofolate reductase (DHFR), and the exogenous factor is an antifolate, e.g., methotrexate. Such a construct is useful in the engineering of amnion derived adherent cells that express IL-1Ra, or a fusion of IL-1Ra and DHFR, upon contact with methotrexate. Such a construct can be used, e.g., in the treatment of rheumatoid arthritis. In this embodiment, the fusion of IL-1Ra and DHFR is translationally upregulated upon exposure to an antifolate such as methotrexate. Therefore, in another specific embodiment, the nucleic acid used to genetically engineer an amnion derived adherent cell can comprise nucleotide sequences encoding a first polypeptide and a second polypeptide, wherein said first and second polypeptides are expressed as a fusion protein that is translationally upregulated in the presence of an exogenous factor. The polypeptide can be expressed transiently or long-term (e.g., over the course of weeks or months). Such a nucleic acid molecule can additionally comprise a nucleotide sequence encoding a polypeptide that allows for positive selection of engineered cells, or allows for visualization of the engineered cells. In another more specific embodiment, the nucleotide sequence encodes a polypeptide that is, e.g., fluorescent under appropriate visualization conditions, e.g., luciferase (Luc). In a more specific embodiment, such a nucleic acid molecule can comprise IL-1Ra-DHFR-IRES-Luc, where IL-1Ra is interleukin-1 receptor antagonist, IRES is an internal ribosomal entry site, and DHFR is dihydrofolate reductase.

4.10.2 Immortalized Amnion Derived Adherent Cell Lines

Mammalian amnion derived adherent cells can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, Ela adenovirus or E7 protein of human papillomavirus. In another embodiment, amnion derived adherent cells can be immortalized using cre-lox recombination, as exemplified for a human pancreatic β-cell line by Narushima, M., et at (Nature Biotechnology, 2005, 23(10:1274-1282).

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells. in one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV*-1}, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the present methods. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized amnion derived adherent cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized adherent cell line prepared as described above. In general, such clonal cell lines may be isolated using standard techniques, such as by limit dilution or using cloning rings, and expanded. Clonal cell lines may generally be fed and passaged as described above.

Conditionally-immortalized human amnion derived adherent cells lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

4.11 Dosages and Routes of Administration

Administration of AMDACs to an individual in need thereof can be by any medically-acceptable route relevant for the disease or condition to be treated. In another specific embodiment of the methods of treatment described above, said AMDACs are administered by bolus injection. In another specific embodiment, said isolated AMDACs are administered by intravenous infusion. In a specific embodiment, said intravenous infusion is intravenous infusion over about 1 to about 8 hours. In another specific embodiment, said isolated AMDACs are administered intracranially. In another specific embodiment, said isolated AMDACs are administered intramuscularly. In another specific embodiment, said isolated AMDACs are administered intraperitoneally. In another specific embodiment, said isolated AMDACs are administered intra-arterially. In a more specific embodiment, said isolated AMDACs are administered within an area of ischemia. In another more specific embodiment, said isolated AMDACs are administered to an area peripheral to an ischemia. In another specific embodiment of the method of treatment, said isolated AMDACs are administered intramuscularly, intradermally, or subcutaneously.

In another specific embodiment of the methods of treatment described above, said AMDACs are administered once to said individual. In another specific embodiment, said isolated AMDACs are administered to said individual in two or more separate administrations. In another specific embodiment, said administering comprises administering between about 1×10⁴ and 1×10⁵ isolated AMDACs, e.g., AMDACs per kilogram of said individual. In another specific embodiment, said administering comprises administering between about 1×10⁵ and 1×10⁶ isolated AMDACs per kilogram of said individual. In another specific embodiment, said administering comprises administering between about 1×10⁶ and 1×10⁷ isolated AMDACs per kilogram of said individual. In another specific embodiment, said administering comprises administering between about 1×10⁷ and 1×10⁸ isolated placental cells per kilogram of said individual. In other specific embodiments, said administering comprises administering between about 1×10⁶ and about 2×10⁶ isolated placental cells per kilogram of said individual; between about 2×10⁶ and about 3×10⁶ isolated placental cells per kilogram of said individual; between about 3×10⁶ and about 4×10⁶ isolated placental cells per kilogram of said individual; between about 4×10⁶ and about 5×10⁶ isolated placental cells per kilogram of said individual; between about 5×10⁶ and about 6×10⁶ isolated placental cells per kilogram of said individual; between about 6×10⁶ and about 7×10⁶ isolated placental cells per kilogram of said individual; between about 7×10⁶ and about 8×10⁶ isolated placental cells per kilogram of said individual; between about 8×10⁶ and about 9×10⁶ isolated placental cells per kilogram of said individual; or between about 9×10⁶ and about 1×10⁷ isolated placental cells per kilogram of said individual. In another specific embodiment, said administering comprises administering between about 1×10⁷ and about 2×10⁷ isolated placental cells per kilogram of said individual to said individual. In another specific embodiment, said administering comprises administering between about 1.3×10⁷ and about 1.5×10⁷ isolated placental cells per kilogram of said individual to said individual. In another specific embodiment, said administering comprises administering up to about 3×10⁷ isolated placental cells per kilogram of said individual to said individual. In a specific embodiment, said administering comprises administering between about 5×10⁶ and about 2×10⁷ isolated placental cells to said individual. In another specific embodiment, said administering comprises administering about 150×10⁶ isolated placental cells in about 20 milliliters of solution to said individual.

In a specific embodiment, said administering comprises administering between about 5×10⁶ and about 2×10⁷ isolated placental cells to said individual, wherein said cells are contained in a solution comprising 10% dextran, e.g., dextran-40, 5% human serum albumin, and optionally an immunosuppressant. In another specific embodiment, said administering comprises administering between about 5×10⁷ and 3×10⁹ isolated placental cells intravenously. In more specific embodiments, said administering comprises administering about 9×10⁸ isolated placental cells or about 1.8×10⁹ isolated placental cells intravenously. In another specific embodiment, said administering comprises administering between about 5×10⁷ and 1×10⁸ isolated placental cells intracranially. In a more specific embodiment, said administering comprises administering about 9×10⁷ isolated placental cells intracranially.

4.12 Differentiation of Amnion Derived Adherent Cells

The amnion derived adherent cells provided herein can be differentiated. In one embodiment, the cell has been differentiated sufficiently for said cell to exhibit at least one characteristic of an endothelial cell, a myogenic cell, or a pericytic cell, e.g., by contacting the cell with vascular endothelial growth factor (VEGF), or as described in Sections 4.11.2, 5.3.3, or 5.3.4, below. In more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, CD9⁺, CD54⁺, CD105⁺, CD200⁺, and VE-cadherin⁻. In other more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, and VEGFR1/Flt-1⁺.

4.12.1 Induction of Angiogenesis

Angiogenesis from the amnion derived adherent cells provided herein can be accomplished as follows. The amnion derived adherent cells, are cultured, e.g., in an endothelial cell medium, e.g., EGM®-2 (Lonza) or a medium comprising 60% DMEM-LG (Gibco), 40% MCDB-201 (Sigma); 2% fetal calf serum (Hyclone Labs.); 1× insulin-transferrin-selenium (ITS); 1× linoleic acid-bovine serum albumin (LA-BSA); 5×10⁻⁹ M dexamethasone (Sigma); 10⁻⁴ M ascorbic acid 2-phosphate (Sigma); epidermal growth factor 10 ng/mL (R&D Systems); and platelet-derived growth factor (PDGF-BB) 10 ng/mL (R&D Systems), to passage 3. The cells are then plated onto MATRIGEL™ or a substrate comprising collagen-1, e.g., in 96-well plates at a density of, e.g., about 1.5×10⁴ cells per well in the same medium or DMEM with FBS (0-5% v/v) comprising vascular endothelial growth factor (VEGF) at, e.g., about 10 to 50 ng per milliliter. Medium can be changed about twice a week. Angiogenesis is evidenced by visual inspection of the cells for sprouting of vessel-like structures and tube formation, visible under a microscope at a magnification of, e.g., 50× to 100×.

4.12.2 Induction of Differentiation into Cardiac Cells

Myogenic (cardiogenic) differentiation of the amnion derived adherent cells provided herein can be accomplished, for example, by placing the cells in cell culture conditions that induce differentiation into cardiomyocytes. A preferred cardiomyocytic medium comprises DMEM/20% CBS supplemented with retinoic acid, 1 μM; basic fibroblast growth factor, 10 ng/mL; and transforming growth factor beta-1, 2 ng/mL; and epidermal growth factor, 100 ng/mL. KnockOut Serum Replacement (Invitrogen, Carlsbad, Calif.) may be used in lieu of CBS. Alternatively, the amnion derived adherent cells are cultured in DMEM/20% CBS supplemented with 1 to 100, e.g., 50 ng/mL Cardiotropin-1 for 24 hours. In another embodiment, amnion derived adherent cells can be cultured 10-14 days in protein-free medium for 5-7 days, then stimulated with human myocardium extract, e.g., produced by homogenizing human myocardium in 1% HEPES buffer supplemented with 1% cord blood serum.

Differentiation can be confirmed by demonstration of cardiac actin gene expression, e.g., by RT/PCR, or by visible beating of the cell. An adherent cell is considered to have differentiated into a cardiac cell when the cell displays one or more of these characteristics.

### 5. EXAMPLES

### 5.1 Example 1

### Treatment of Schizophrenia Using Amnion Derived Adherent Cells

5.1.1 Patient 1

Patient 1 presents with self-described auditory and tactile hallucinations, and appears to have no or very reduced emotional response to questions (flat affect). Patient 1 has lost his job within the last three weeks due to his mental problems, which according to him began approximately three months earlier. Patient 1 reports no family history of schizophrenia. After voluntarily checking into a mental health facility, Patient 1 is observed for one week, during which time his self-reported symptoms are confirmed. Per the Diagnostic and Statistical Manual of Mental Disorders, Fourth Edition (DSM-IV), Patient 1 is diagnosed with schizophreniform disorder. As an alternative to antipsychotic drug therapy, Patient 1 is administered 2.5×10⁹ CD49f⁺ OCT-4⁻ AMDACs by intravenous infusion, and is observed for an additional two weeks. Patient 1 reports a reduction in the number and intensity of the hallucinations, and appears to have increased affect.

5.1.2 Patient 2

Patient 2, a 45-year old 75 kg male, presents with self-described auditory and tactile hallucinations, and appears to have no or very reduced emotional response to questions (flat affect). Patient 2 has lost his job within the last three weeks due to his mental problems, which according to him began approximately three months earlier. Patient 2 reports a second cousin who was diagnosed in the last two years with schizophrenia. After voluntarily checking into a mental health facility, Patient 2 is observed for one week, during which time his self-reported symptoms are confirmed. Per the Diagnostic and Statistical Manual of Mental Disorders, Fourth Edition (DSM-IV), Patient 2 is diagnosed with schizophreniform disorder. As an alternative to antipsychotic drug therapy, Patient 2 is administered 2.5×10⁹ CD49f⁺ OCT-4⁻ AMDACs by intravenous infusion, and is observed for an additional two weeks. Patient 2 reports a reduction in the number and intensity of the hallucinations, and appears to have increased affect.

Within two months after initial treatment, Patient 2 reports a recurrence of the auditory and tactile hallucinations, and again appears to have a reduced emotional response. Patient 2 is administered a second dose of 2.5×10⁹ CD49f⁺ OCT-4⁻ AMDACs, and is monitored for an additional two weeks, during which time Patient 2 reports a reduction in the number and intensity of the hallucinations, and appears, again, to have increased affect.

5.1.3 Patient 3

Patient 3, a 37 year old 80 kg male, presents with disordered speech, auditory hallucinations that, according to Patient 3, comment on his actions (including his initial conversation with an attending psychiatrist, and recurring delusions that family members are planning to attack him. According to family members, his symptoms first began to manifest seven months earlier, and have rapidly progressed within the last month. Patient 3 is additionally characterized by family members as having lost interest in personal hygiene during that period, particularly within the last month. Family members report no known occurrence of schizophrenia in the extended family. After voluntarily checking into a mental health facility, Patient 3 is observed for one week, during which time his self-reported and family-reported symptoms are confirmed. Per the Diagnostic and Statistical Manual of Mental Disorders, Fourth Edition (DSM-IV), Patient 3 is diagnosed with schizophrenia. Patient 3 is determined to be mentally unable to consent to therapy; as such, consent for therapy is provided by Patient 3's spouse.

As an alternative to antipsychotic drug therapy, Patient 3 is administered 2.5×10⁹ CD49f⁺ OCT-4⁻ AMDACs by intravenous infusion, and is observed for an additional two weeks. At the end of the two weeks, Patient 3 is determined by a psychiatrist to have significantly more ordered speech than when first admitted, and a reduction in the number and intensity of the auditory hallucinations.

5.1.4 Patient 4

Patient 4 a 37 year old 80 kg male, presents with disordered speech, auditory hallucinations that, according to Patient 4, comment on his actions (including his initial conversation with an attending psychiatrist, and recurring delusions that family members are planning to attack him. According to family members, his symptoms first began to manifest seven months earlier, and have rapidly progressed within the last month. Patient 4 is additionally characterized by family members as having lost interest in personal hygiene during that period, particularly within the last month. Family members report no known occurrence of schizophrenia in the extended family. After voluntarily checking into a mental health facility, Patient 4 is observed for one week, during which time his self-reported and family-reported symptoms are confirmed. Per the Diagnostic and Statistical Manual of Mental Disorders, Fourth Edition (DSM-IV), Patient 4 is diagnosed with schizophrenia. Patient 4 is determined to be mentally unable to consent to therapy; as such, consent for therapy is provided by Patient 4's spouse.

Patient 4 is placed on risperidone (RISPERDAL®) at an initial dose of 2 mg/day, which is increased over four days to 4 mg/day. As an adjunct to risperidone therapy, after two weeks on risperidone, Patient 4 is administered 2.5×10⁹ CD49f⁺ OCT-4⁻ AMDACs by intravenous infusion, and is observed for an additional two weeks. At the end of the two weeks, Patient 4 is determined by a psychiatrist to have significantly more ordered speech than when first admitted, and a reduction in the number and intensity of the auditory hallucinations.

5.1.5 Patient 5

Patient 5, a 25 year old 80 kg male, presents with disordered speech, including pressured speech, auditory hallucinations, and delusions that he has been designated as a messianic figure. According to family members, his symptoms first began to manifest a year earlier, five months after which Patient 5 was involuntarily committed by his family. Patient 5 is additionally characterized by family members as having lost interest in personal hygiene during the period prior to commitment. Family members report no known occurrence of schizophrenia in the extended family. Patient 5 was diagnosed upon commitment with schizophrenia per the Diagnostic and Statistical Manual of Mental Disorders, Fourth Edition (DSM-IV).

Patient 5 was initially placed on risperidone (RISPERDAL®) at an initial dose of 2 mg/day, which was increased over four days to 4 mg/day. On failure of risperidone to resolve any of the symptoms satisfactorily, Patient 5 is administered 2.5×10⁹ CD49f⁺ OCT-4⁻ AMDACs by intravenous infusion, and is observed for two weeks thereafter. At the end of the two weeks, Patient 5 is determined by a psychiatrist to have significantly more ordered speech than when first admitted, and a reduction in the number and intensity of the auditory hallucinations. Patient 5 is readministered AMDACs at the same dosage during the commitment period at intervals of 3-5 weeks, or as needed based upon relapse (noticeable worsening of one or more symptoms).

5.1.6 Patient 6

Patient 6, a 25 year old 80 kg male, presents with disordered speech, including pressured speech, auditory hallucinations, and delusions that he has been designated as a messianic figure. According to family members, his symptoms first began to manifest a year earlier, five months after which Patient 6 was involuntarily committed by his family. Patient 6 is additionally characterized by family members as having lost interest in personal hygiene during the period prior to commitment. Family members report no known occurrence of schizophrenia in the extended family. Patient 6 was diagnosed upon commitment with schizophrenia per the Diagnostic and Statistical Manual of Mental Disorders, Fourth Edition (DSM-IV).

Patient 6 was initially placed on risperidone (RISPERDAL®) at an initial dose of 2 mg/day, which was increased over four days to 4 mg/day. Risperidone therapy produced satisfactory results for three months, during which time Patient 6 was discharged. However, despite maintenance of risperidone therapy, Patient 6's symptoms re-emerged, and Patient 6 was re-committed. Patient 6 is administered 2.5×10⁹ CD49f⁺ OCT-4⁻ AMDACs by intravenous infusion, and is observed for two weeks thereafter. At the end of the two weeks, Patient 6 is determined by a psychiatrist to have significantly more ordered speech than when first admitted, and a reduction in the number and intensity of the auditory hallucinations.

### 5.2 Example 2

### Isolation and Expansion of Adherent Cells from Amniotic Membrane

This Example demonstrates the isolation and expansion of amnion derived adherent cells.

5.2.1 Isolation

Amnion derived adherent cells were isolated from amniotic membrane as follows. Amnion/chorion were cut from the placenta, and amnion was manually separated from the chorion. The amnion was rinsed with sterile PBS to remove residual blood, blood clots and other material. Sterile gauze was used to remove additional blood, blood clots or other material that was not removed by rinsing, and the amnion was rinsed again with PBS. Excess PBS was removed from the membrane, and the amnion was cut with a scalpel into 2″ by 2″ segments. For epithelial cell release, a processing vessel was set up by connecting a sterile jacketed glass processing vessel to a circulating 37° C. water bath using tubing and connectors, and set on a stir plate. Trypsin (0.25%, 300 mL) was warmed to 37° C. in the processing vessel; the amnion segments were added, and the amnion/trypsin suspension was agitated, e.g., at 100 RPM-150 RPM at 37° C. for 15 minutes. A sterile screening system was assembled by placing a sterile receptacle on a sterile field next to the processing vessel and inserting a sterile 75 μm to 125 μm screen into the receptacle (Millipore, Billerica, Mass.). After agitating the amnion segments for 15 minutes, the contents of the processing vessel were transferred to the screen, and the amnion segments were transferred, e.g., using sterile tweezers back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel, and the trypsin solution containing the epithelial cells was discarded. The amnion segments were agitated in PBS/5% FBS (1:1 ratio of amnion to PBS/5% FBS solution by volume) at 37° C. for approximately 2-5 minutes to neutralize the trypsin. A fresh sterile screen system was assembled. After neutralizing the trypsin, the contents of the processing vessel were transferred to the new screen, and the amnion segments were transferred back into the processing vessel. Room temperature, sterile PBS (400 mL) was added to the processing vessel, and the contents of the processing vessel were agitated for approximately 2-5 minutes. The screen was rinsed with approximately 100-150 ml, of PBS. After agitation, the contents of the processing vessel were transferred to the screen; the processing flask was rinsed with PBS, and the PBS solution was discarded. The processing vessel was then filled with 300 mL of pre-warmed DMEM, and the amnion segments were transferred into the DMEM solution.

For release of the amnion derived adherent cells, the treated amniotic membrane was further treated with collagenase as follows. A sterile collagenase stock solution (500 U/mL) was prepared by dissolving the appropriate amount of collagenase powder (varied with the activity of the collagenase lot received from the supplier) in DMEM. The solution was filtered through a 0.22 μm filter and dispensed into individual sterile containers. CaCl₂ solution (0.5 mL, 600 mM) was added to each 100 mL dose, and the doses were frozen. Collagenase (100 mL) was added to the amnion segments in the processing vessel, and the processing vessel was agitated for 30-50 minutes, or until amnion digestion was complete by visual inspection. After amnion digestion was complete, 100 mL of pre-warmed sterile PBS/5% FBS was added to the processing vessel, and the processing vessel was agitated for an additional 2-3 minutes. Following agitation, the contents of the flask were transferred to a sterile 60 μm screen, and the liquid was collected by vacuum filtration. The processing vessel was rinsed with 400 mL of PBS, and the PBS solution was sterile-filtered. The filtered cell suspension was then centrifuged at 300×g for 15 minutes at 20° C., and the cell pellets were resuspended in pre-warmed PBS/2% FBS (approximately 10 mL total).

5.2.2 Establishment

Freshly isolated angiogenic amniotic cells were added to growth medium containing 60% DMEM-LG (Gibco); 40% MCBD-201 (Sigma); 2% FBS (Hyclone Labs), 1× insulin-transferrin-selenium (ITS); 10 ng/mL linoleic acid-bovine serum albumin (LA-BSA); 1 n-dexamethasone (Sigma); 100 μM ascorbic acid 2-phosphate (Sigma); 10 ng/mL epidermal growth factor (R & D Systems); and 10 ng/mL platelet-derived growth factor (PDGF-BB) (R & D Systems) and were plated in a T-Flask at a seeding density of 10,000 cells per cm². The culture device(s) were then incubated at 37° C., 5% CO₂ with >90% humidity. Cellular attachment, growth, and morphology were monitored daily. Non-adherent cells and debris were removed by medium exchange. Medium exchange was performed twice per week. Adherent cells with typical fibroblastoid/spindle shape morphology appeared at several days after initial plating. When confluency reached 40%-70% (at 4-11 days after initial plating), the cells were harvested by trypsinization (0.25% trypsin—EDTA) for 5 minutes at room temperature (37° C.). After neutralization with PBS-5% FBS, the cells were centrifuged at 200-400 g for 5-15 minutes at room temperature, and then were resuspended in growth medium. At this point, an AMDAC line was considered to be successfully established at the initial passage. Initial passage amnion derived adherent cells were, in some cases, cryopreserved or expanded.

5.2.3 Culture Procedure

Amnion derived adherent cells were cultured in the growth medium described above and seeded at a density of 2000-4000 per cm² in an appropriate tissue culture—treated culture device(s). The culture device(s) were then incubated at 37° C., 5% CO₂ with >90% humidity. During culture, AMDACs would adhere and proliferate. Cellular growth, morphology, and confluency were monitored daily. Medium exchange was performed twice a week to replenish fresh nutrients if the culture extended to 5 days or more. When confluency reached 40%-70% (at 3-7 days after seeding), the cells were harvested by trypsinization (0.05%-0.25% trypsin—EDTA) for 5 minutes at room temperature (37° C.). After neutralization with PBS-5% FBS, the cells were centrifuged at 200-400 g for 5-15 minutes at room temperature, then were resuspended in growth medium.

AMDACs isolated and cultured in this manner typically produced 33530+/−15090 colony-forming units (fibroblast) (CFU-F) out of 1×10⁶ cells plated.

### 5.3 Example 3

### Phenotypic Characterization of Amnion Derived Adherent Cells

5.3.1 Gene and Protein Expression Profiles

This Example describes phenotypic characterization of amnion derived adherent cells, including characteristic cell surface marker, mRNA, and proteomic expression.

Sample Preparation:

Amnion derived adherent cells were obtained as described in Example 1. The cells at passage 6 were grown to approximately 70% confluence in growth medium as described in Example 1, above, trypsinized, and washed in PBS. NTERA-2 cells (American Type Culture Collection, ATCC Number CRL-1973) were grown in DMEM containing 4.5 g/L glucose, 2 mM glutamine and 10% FBS. Nucleated cell counts were performed to obtain a minimum of 2×10⁶ to 1×10⁷ cells. The cells were then lysed using a Qiagen RNeasy kit (Qiagen, Valencia, Calif.), utilizing a QlAshredder, to obtain the lysates. The RNA isolation was then performed using a Qiagen RNeasy kit. RNA quantity and quality were determined using a Nanodrop ND1000 spectrophotometer, 25 ng/μL of RNA/reaction. The cDNA reactions were prepared using an Applied Biosystems (Foster City, Calif.) High Capacity cDNA Archive Kit. Real time PCR reactions were performed using TAQMAN® universal PCR master mixes from Applied Biosystems. Reactions were run in standard mode on an Applied Biosystems 7300 Real time PCR system for 40 cycles.

Sample Analysis and Results:

Using the real time PCR methodology and specific TAQMAN® gene expression probes and/or the TAQMAN® human angiogenesis array (Applied Biosystems), cells were characterized for expression of stem cell-related, angiogenic and cardiomyogenic markers. Results were expressed either as the relative expression of a gene of interest in comparison to the pertinent cell controls, or the relative expression (delta Ct) of the gene of interest in comparison to a ubiquitously expressed housekeeping gene (for example, GAPDH, 18S, or GUSB).

Amnion derived adherent cells expressed various, stem-cell related, angiogenic and cardiomyogenic genes and displayed a relative absence of OCT-4 expression in comparison to NTERA-2 cells. Table 1 summarizes the expression of selected angiogenic, cardiomyogenic, and stem cell genes. AMDACs were further found to lack expression of the stem cell-related genes POU5F1 (OCT-4), NANOG, SOX2, NES, DNMT3B, and TERT.

TABLE 1
Gene expression profile of amnion derived
adherent cells as determined by RT-PCR.

| AMDAC Marker | Positive | Negative |
|---|---|---|
| ACTA2 | X | |
| ACTC1 | X | |
| ADAMTS1 | X | |
| AMOT | X | |
| ANG | X | |
| ANGPT1 | X | |
| ANGPT2 | X | |
| ANGPT4 | | X |
| ANGPTL1 | X | |
| ANGPTL2 | X | |
| ANGPTL3 | | X |
| ANGPTL4 | X | |
| BAI1 | X | |
| BGLAP | | X |
| c-myc | X | |
| CD31 | | X |
| CD34 | | X |
| CD44 | X | |
| CD140a | X | |
| CD140b | X | |
| CD200 | X | |
| CD202b | X | |
| CD304 | X | |
| CD309 (VEGFR2/KDR) | X | |
| CDH5 | | X |
| CEACAM1 | X | |
| CHGA | X | |
| COL15A1 | X | |
| COL18A1 | X | |
| COL4A1 | X | |
| COL4A2 | X | |
| COL4A3 | X | |
| Connexin-43 | X | |
| CSF3 | X | |
| CTGF | X | |
| CXCL10 | | X |
| CXCL12 | X | |
| CXCL2 | X | |
| DLX5 | | X |
| DNMT3B | X | |
| ECGF1 | X | |
| EDG1 | X | |
| EDIL3 | X | |
| ENPP2 | X | |
| EPHB2 | X | |
| F2 | X | |
| FBLN5 | X | |
| FGA | | X |
| FGF1 | X | |
| FGF2 | X | |
| FGF4 | | X |
| FIGF | X | |
| FLT3 | | X |
| FLT4 | X | |
| FN1 | X | |
| FOXC2 | X | |
| Follistatin | X | |
| Galectin-1 | X | |
| GRN | X | |
| HEY1 | X | |
| HGF | X | |
| HLA-G | | X |
| HSPG2 | X | |
| IFNB1 | X | |
| IFNG | | X |
| IL-8 | X | |
| IL-12A | X | |
| ITGA4 | X | |
| ITGAV | X | |
| ITGB3 | X | |
| KLF-4 | X | |
| LECT1 | | X |
| LEP | | X |
| MDK | X | |
| MMP-13 | | X |
| MMP-2 | X | |
| MYOZ2 | X | |
| NANOG | | X |
| NESTIN | | X |
| NRP2 | X | |
| PDGFB | X | |
| PF4 | X | |
| PGK1 | X | |
| PLG | | X |
| POU5F1 (OCT-4) | | X |
| PRL | | X |
| PROK1 | | X |
| PROX1 | X | |
| PTN | X | |
| SEMA3F | X | |
| SERPINB5 | X | |
| SERPINC1 | X | |
| SERPINF1 | X | |
| SOX2 | | X |
| TERT | | X |
| TGFA | X | |
| TGFB1 | X | |
| THBS1 | X | |
| THBS2 | X | |
| TIE1 | X | |
| TIMP2 | X | |
| TIMP3 | X | |
| TNF | X | |
| TNFSF15 | X | |
| TNMD | | X |
| TNNC1 | X | |
| TNNT2 | X | |
| VASH1 | X | |
| VEGF | X | |
| VEGFB | X | |
| VEGFC | X | |
| VEGFR1/FLT-1 | X | |
| XLKD1 | | X |

Column “mRNA” indicates that the presence or absence of mRNA for particular markers were determined in each instance.

In a separate experiment, AMDACs were additionally found to express genes for Aryl hydrocarbon receptor nuclear translocator 2 (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB1R), and hepatoycte growth factor receptor (HGFR/c-met).

5.3.2 Flow Cytometry for Evaluation of Angiogenic Potency of Amnion Derived Adherent Cells

Flow cytometry was used as a method to quantify phenotypic markers of amnion derived adherent cells to define the identity of the cells. Cell samples were obtained from frozen stocks. Prior to thaw and during reagent preparation, cell vials were maintained on dry ice. Subsequently, samples were thawed rapidly using a 37° C. water bath. Pre-freeze cell counts were used for calculations for initial post-thaw cell number-dependent dilutions. Briefly, cryovials were thawed in a 37° C. water bath for approximately 30 seconds with gentle agitation. Immediately following thawing, approximately 100-200 μL of cold (2 to 8° C.) thawing solution (PBS with 2.5% albumin and 5% Gentran 40) was added to the cryovial and mixed. After gentle mixing, the total volume in the cryovials was transferred into a 15 mL conical tube containing an equal volume of cold (2 to 8° C.) thawing solution. The cells were centrifuged in a conical tube at 400 g for 5 minutes at room temperature before removing the supernatant. The residual volume was measured with a pipette (estimation); the residual volume and cell pellet were resuspended at room temperature in 1% FBS in PBS to achieve a cell concentration of 250×10³ cells/100 μL buffer. For example, 1×10⁶ cells would be resuspended in 400 μL 1% FBS. The cell suspension was placed into pre-labeled 5 mL FACS tubes (Becton Dickinson (BD), Franklin Lakes, N.J.). For each primary antibody isotype, 100 μL of cell suspension was aliquoted into one isotype control tube. Prior to phenotype analysis, the concentrations of all antibodies were optimized to achieve good signal to noise ratios and adequate detection of CD antigens across a potential four-log dynamic range. The volume of each isotype and sample antibody that was used to stain each sample was determined. To standardize the amount of antibody (in μg) in the isotype and sample tubes, the concentration of each antibody was calculated as (1/actual antibody concentration (μg/μL))×(desired final quantity of antibody in μg for 2.5×10⁵ cells)=#μL of antibody added. A master mix of antibodies for both the isotype and the sample was made with the appropriate amount of antibody added to each tube. The cells were stained for 15-20 minutes at room temperature in the dark. After staining, unbound antibody in each sample was removed by centrifugation (400 g×5 minutes) followed by washing using 2 mL 1% FBS PBS (room temperature) before resuspension in 150 μL of room temperature 1% FBS PBS. The samples were then analyzed on Becton Dickinson FACSCalibur, FACSCantoI or BD FACSCantoII flow cytometers prepared for use per manufacturer's instructions. Multi-parametric flow cytometry data sets (side scatter (SSC), forward scatter (FSC) and integrated fluorescence profiles (FL)) were acquired without setting on-the-fly instrument compensation parameters. Compensation parameters were determined after acquisition using the FACSDiva software according to the manufacturer's instructions. These instrument settings were applied to each sample. Fluorophore conjugates used in these studies were Allophycocyanin (APC), AlexaFluor 647 (AF647), Fluorescein isothiocyanate (FITC), Phycoerythrin (PE) and Peridinin chlorophyll protein (PerCP), all from BD Biosciences. Table 2 summarizes the expression of selected cell-surface markers, including angiogenic markers.

TABLE 2
Cell surface marker expression in amnion derived
adherent cells as determined by flow cytometry.

| | | | Immunolocalization |
|---|---|---|---|
| AMDAC Marker | Positive | Negative | Flow Cytometry |
| CD6 | | X | X |
| CD9 | X | | X |
| CD10 | X | | X |
| CD31 | | X | X |
| CD34 | | X | X |
| CD44 | X | | X |
| CD45 | | X | X |
| CD49b | X | | X |
| CD49c | X | | X |
| CD49d | X | | X |
| CD54 | X | | X |
| CD68 | X | | X |
| CD90 | X | | X |
| CD98 | X | | X |
| CD105 | X | | X |
| CD117 | | X | X |
| CD133 | | X | X |
| CD143 | | X | X |
| CD144 (VE-cadherin) | | X | X |
| CD146 | | X | X |
| CD166 | X | | X |
| CD184 | | X | X |
| CD200 | X | | X |
| CD202b | X | | X |
| CD271 | | X | X |
| CD304 | X | | X |
| CD309 (VEGFR2/KDR) | X | | X |
| CD318 | X | | X |
| CD349 | X | | X |
| CytoK | X | | X |
| HLA-ABC + | X | | X |
| B2 Micro+ | | | |
| Invariant Chain + | | X | X |
| HLA-DR-DP-DQ+ | | | |
| PDL-1 | X | | X |
| VEGFR1/FLT-1 | X | | X |
| | | | X |

Column “Immunolocalization Flow Cytometry” indicates that the presence or absence of particular markers were determined by immunolocalization, specifically flow cytometry.

In another experiment, AMDAC cells were labeled with anti-human CD49f (Clone GoH3, phycoerythrin-conjugated; BD Pharmingen Part No. 555736), and analyzed by flow cytometry. Approximately 96% of the AMDACs labeled with anti-CD49f (that is, were CD49f⁺).

In other experiments, AMDACs were additionally found by immunolocalization to express CD49a, CD106, CD119, CD130, c-met (hepatocyte growth factor receptor; HGFR), CXC chemokine receptor 1 (CXCR1), PDGFRA, and PDGFRB by immunolocalization. AMDACs were also found, by immunolocalization, to lack expression of CD49e, CD62E, fibroblast growth factor receptor 3 (FGFR3), tumor necrosis factor receptor superfamily member 12A (TNFRSF12A), insulin-like growth factor 1 receptor (IGF-1R), CXCR2, CXCR3, CXCR4, CXCR6, chemokine receptor 1 (CCR1), CCR2, CCR3, CCR4, CCR5, CCR6, CCR7, CCR8, CCR9, epidermal growth factor receptor (EGF-R), insulin receptor (CD220), interleukin receptor 4 (IL4-R; CD124), IL6-R (CD126), TNF-R1a and 1b (CD120a, b), and erbB2/Her2.

5.3.3 Immunohistochemistry (IHC)/Immunofluorochemistry (IFC) for Evaluation of Angiogenic Potency of Amnion Derived Adherent Cells

Amnion derived adherent cells from passage 6 were grown to approximately 70% confluence on 4-well chamber slides and fixed with a 4% formalin solution for 30 minutes each. After fixation, the slides were rinsed with PBS two times for 5 minutes. The slides were then incubated with 10% normal serum from the same host as the secondary antibody, 2× casein, and 0.3% Triton X100 in PBS, for 20 minutes at room temperature in a humid chamber. Excess serum was blotted off and the slides were incubated with primary antibody (goat polyclonal IgG (Santa Cruz; Santa Cruz, Calif.) in a humidified chamber. Time and temperature for incubations were determined by selecting the optimal conditions for the antibody being used. In general, incubation times were 1 to 2 hours at 37° C. or overnight at 4° C. The slides were then rinsed with PBS three times for 5 minutes each and incubated for 20-30 minutes at room temperature in a humid chamber with fluorescent-conjugated anti-immunoglobulin secondary antibody directed against the host of the primary antibody (rabbit anti-goat antibody (Santa Cruz)). Thereafter, the slides were rinsed with PBS three times for 5 minutes each, mounted with a coverslip utilizing DAPI VECTASHIELD® (Vector Labs) mounting solution to counterstain nuclei. Cell staining was visualized utilizing a Nikon fluorescence microscope. All pictures were taken at equal exposure time normalized against the background of the corresponding isotype (goat IgG (Santa Cruz)). Table 3 summarizes the results for the expression of angiogenic proteins by amnion derived adherent cells.

TABLE 3
Angiogenic markers present or absent
on amnion derived adherent cells.

| AMDAC Marker | Positive | Negative |
|---|---|---|
| CD31 | | X |
| CD34 | | X |
| (VEGFR2/KDR) | X | |
| Connexin-43 | X | |
| Galectin-1 | X | |
| TEM-7 | X | |

Amnion derived adherent cells expressed the angiogenic marker tumor endothelial marker 7 (TEM-7), one of the proteins shown in Table 3.

5.3.4 Membrane Proteomics for Evaluation of Angiogenic Potency of Amnion Derived Adherent Cells

Membrane Protein Purification:

Cells at passage 6 were grown to approximately 70% confluence in growth medium, trypsinized, and washed in PBS. The cells were then incubated for 15 minutes with a solution containing protease inhibitor cocktail (P8340, Sigma Aldrich, St. Louis, Mo.) prior to cell lysis. The cells were then lysed by the addition of a 10 mM HCl solution (thus avoiding the use of detergents) and centrifuged for 10 minutes at 400 g to pellet and remove the nuclei. The post-nuclear supernatant was transferred to an ultracentrifugation tube and centrifuged using a WX80 ultracentrifuge with a T-1270 rotor (Thermo Fisher Scientific, Asheville, N.C.) at 100,000 g for 150 minutes generating a membrane protein pellet.

Generation, Immobilization and Digestion of Proteoliposomes:

The membrane protein pellet was washed several times using Nanoxis buffer (10 mM Tris, 300 mM NaCl, pH 8). The membrane protein pellet was suspended in 1.5 mL of Nanoxis buffer and then tip-sonicated using a VIBRA-CELL™ VC505 ultrasonic processor (Sonics & Materials, Inc., Newtown, Conn.) for 20 minutes on ice. The size of the proteoliposomes was determined by staining with FM1-43 dye (Invitrogen, Carlsbad, Calif.) and visualization with fluorescence microscopy. The protein concentration of the proteoliposome suspension was determined by a BCA assay (Thermo Scientific). The proteoliposomes were then injected onto an LPITmFlow Cell (Nanoxis AB, Gothenburg, Sweden) using a standard pipette tip and allowed to immobilize for 1 hour. After immobilization, a series of washing steps were carried out and trypsin at 5 μg/mL (Princeton Separations, Adelphi, N.J.) was injected directly onto the LPI™ Flow Cell. The chip was incubated overnight at 37° C. and the tryptic peptides were eluted from the LPI™ chip and then desalted using a Sep-Pak cartridge (Waters Corporation, Milford, Mass.).

LTQ Linear Ion Trap LC/MS/MS Analysis:

Each tryptic digest sample was separated on a 0.2 mm×150 mm 3 μm 200 Å MAGIC C18 column (Michrom Bioresources, Inc., Auburn, Calif.) that was interfaced directly to an axial desolvation vacuum-assisted nanocapillary electrospray ionization (ADVANCE) source (Michrom Bioresources, Inc.) using a 180 minute gradient (Buffer A: Water, 0.1% Formic Acid; Buffer B: Acetonitrile, 0.1% Formic Acid). The ADVANCE source achieves a sensitivity that is comparable to traditional nanoESI while operating at a considerably higher flow rate of 3 μL/min. Eluted peptides were analyzed on an LTQ linear ion trap mass spectrometer (Thermo Fisher Scientific, San Jose, Calif.) that employed ten data-dependent MS/MS scans following each full scan mass spectrum. Seven analytical replicate datasets were collected for each biological sample.

Bioinformatics:

Seven RAW files corresponding to the 7 analytical replicate datasets that were collected for each cell line were searched as a single search against the IPI Human Database using an implementation of the SEQUEST algorithm on a Sorcerer Solo™ workstation (Sage-N Research, San Jose, Calif.). A peptide mass tolerance of 1.2 amu was specified, oxidation of methionine was specified as a differential modification, and carbamidomethylation was specified as a static modification. Scaffold software implementation of the Trans-Proteomic Pipeline (TPP) was used to sort and parse the membrane proteomic data. Proteins were considered for analysis if they were identified with a peptide probability of 95%, protein probability of 95% and 1 unique peptide. Comparisons between membrane proteomic datasets were made using custom Perl scripts developed in-house.

Results:

As shown in Table 4, amnion derived adherent cells expressed various angiogenic and cardiomyogenic markers.

TABLE 4
Cardiomyogenic or angiogenic markers expressed
by amnion derived adherent cells.

| AMDAC Marker | Positive | Negative |
|---|---|---|
| Activin receptor type IIB | X | |
| ADAM 17 | X | |
| Alpha-actinin 1 | X | |
| Angiotensinogen | X | |
| Filamin A | X | |
| Macrophage acetylated | X | |
| LDL receptor I and II | | |
| Megalin | X | |
| Myosin heavy chain non | X | |
| muscle type A | | |
| Myosin-binding protein | X | |
| C cardiac type | | |
| Wnt-9 | X | |

5.3.5 Secretome Profiling for Evaluation of Angiogenic Potency of Amnion Derived adherent cells

Protein Arrays:

Amnion derived adherent cells at passage 6 were plated at equal cell numbers in growth medium and conditioned media were collected after 4 days. Simultaneous qualitative analysis of multiple angiogenic cytokines/growth factors in cell-conditioned media was performed using RayBiotech Angiogenesis Protein Arrays (Norcross, Ga.). In brief, protein arrays were incubated with 2 mL 1× Blocking Buffer (Ray Biotech) at room temperature for 30 minutes (min) to block membranes. Subsequently, the Blocking Buffer was decanted and the membranes were incubated with 1 mL of sample (growth medium conditioned by the respective cells for 4 days) at room temperature for 1 to 2 hours. The samples were then decanted and the membranes were washed 3×5 min with 2 mL of 1× Wash Buffer I (Ray Biotech) at room temperature with shaking. Then, the membranes were washed 2×5 min with 2 mL of 1× Wash Buffer II (Ray Biotech) at room temperature with shaking Thereafter, 1 mL of diluted biotin-conjugated antibodies (Ray Biotech) was added to each membrane and incubated at room temperature for 1-2 hours and washed with the Wash Buffers as described above. Diluted HRP-conjugated streptavidin (2 mL) was then added to each membrane and the membranes were incubated at room temperature for 2 hours. Finally, the membranes were washed again, incubated with the ECL™ detection kit (Amersham) according to specifications and the results were visualized and analyzed using the Kodak Gel Logic 2200 Imaging System.

ELISAs:

Quantitative analysis of single angiogenic cytokines/growth factors in cell-conditioned media was performed using commercially available kits from R&D Systems (Minneapolis, Minn.). In brief, ELISA assays were performed according to manufacturer's instructions and the amount of the respective angiogenic growth factors in the conditioned media was normalized to 1×10⁶ cells. Amnion derived adherent cells (n=6) exhibited approximately 4500 pg VEGF per million cells and approximately 17,200 pg IL-8 per million cells.

TABLE 5
ELISA results for angiogenic markers

| AMDAC Marker | Positive | Negative |
|---|---|---|
| ANG | X | |
| EGF | X | |
| ENA-78 | X | |
| FGF2 | X | |
| Follistatin | X | |
| G-CSF | X | |
| GRO | X | |
| HGF | X | |
| IL-6 | X | |
| IL-8 | X | |
| Leptin | X | |
| MCP-1 | X | |
| MCP-3 | X | |
| PDGFB | X | |
| PLGF | X | |
| Rantes | X | |
| TGFB1 | X | |
| Thrombopoietin | X | |
| TIMP1 | X | |
| TIMP2 | X | |
| uPAR | X | |
| VEGF | X | |
| VEGFD | X | |

In a separate experiment, AMDACs were confirmed to also secrete angiopoietin-1, angiopoietin-2, PECAM-1 (CD31; platelet endothelial cell adhesion molecule), laminin and fibronectin.

5.3.6 AMDAC MicroRNA Expression Confirms Angiogenic Activity

This Example demonstrates that AMDACs express higher levels of certain micro-RNAs (miRNAs), and lower levels of certain other miRNAs, each of which correlated with angiogenic function, than bone marrow-derived mesenchymal stem cells.

It is known that pro-angiogenic miR-296 regulates angiogenic function through regulating levels of growth factor receptors. For example, miR-296 in endothelial cells contributes significantly to angiogenesis by directly targeting the hepatocyte growth factor-regulated tyrosine kinase substrate (HGS) mRNA, leading to decreased levels of HGS and thereby reducing HGS-mediated degradation of the growth factor receptors VEGFR2 and PDGFRb. See Wiirdinger et al., Cancer Cell 14:382-393 (2008). In addition, miR-15b and miR-16 have been shown to control the expression of VEGF, a key pro-angiogenic factor involved in angiogenesis, and that hypoxia-induced reduction of miR-15b and miR-16 contributes to an increase in VEGF, a pro-angiogenic cytokine See Kuelbacher et al., Trends in Pharmacological Sciences, 29(1):12-15 (2007).

AMDACs were prepared as described in Example 1, above. AMDACs and BM-MSC cells (used as a comparator) were subjected to microRNA (miRNA) preparation using a MIRVANA™ miRNA Isolation Kit (Ambion, Cat#1560). 0.5×10⁶ to 1.5×10⁶ cells were disrupted in a denaturing lysis buffer. Next, samples were subjected to acid-phenol+chloroform extraction to isolate RNA highly enriched for small RNA species. 100% ethanol was added to bring the samples to 25% ethanol. When this lysate/ethanol mixture was passed through a glass fiber filter, large RNAs were immobilized, and small RNA species were collected in the filtrate. The ethanol concentration of the filtrate was then increased to 55%, and the mixture was passed through a second glass fiber filter where the small RNAs became immobilized. This RNA was washed, and eluted in a low ionic strength solution. The concentration and purity of the recovered small RNA was determined by measuring its absorbance at 260 and 280 nm.

AMDACs were found to express the following angiogenic miRNA: miR-17-3p, miR-18a, miR-18b, miR-19b, miR-92, miR-20a, miR-20b, (members of the of the angiogenic miRNA cluster 17-92), miR-296, miR-221, miR-222, miR-15b, miR-16. AMDACs were also found to express higher levels of the following angiogenic miRNA when compared to bone marrow-derived mesenchymal stem cells (BM-MSCs): miR-17-3p, miR-18a, miR-18b, miR-19b, miR-92 (members of the of the angiogenic miRNA cluster 17-92), miR-296. These results correlate well with the observation that AMDACs express high levels of VEGFR2/KDR (see above). Conversely, AMDACs were found to express lower levels of the following angiogenic miRNA when compared to BM-MSCs: miR-20a, miR-20b, (members of the of the angiogenic miRNA cluster 17-92), miR-221, miR-222, miR-15b, miR-16. The reduced expression of miR-15b and miR-16 correlated with the higher levels of expression of VEGF seen in AMDACs.

### 5.4 Example 4

### AMDACs Exhibit Neuroprotective Effect

This Example demonstrates that AMDACs have a neuroprotective effect in low-oxygen and low-glucose conditions using an oxygen-glucose deprivation (OGD) insult assay, and reduce reactive oxygen species. As such, these results indicate that AMDACs would be useful in treating ischemic conditions such as stroke or peripheral vascular disease, and would protect against reperfusion injuries resulting from ischemic conditions.

Human neurons (ScienCell, catalog #1520) were cultured as per manufacturer's recommendations. Briefly, culture vessels were coated with Poly-L-Lysine (2 μg/mL) in sterile distilled water for 1 hour at 37° C. The vessel was washed with double distilled H₂O three times. Neuron Medium (ScienCell) was added to vessel and equilibrated to 37° C. in an incubator. Neurons were thawed, and added directly into the vessels without centrifugation. During subsequent culture, medium was changed the day following culture initiation, and every other day thereafter. The neurons were typically ready for insult by day 4.

OGD medium (Dulbecco's Modified Eagle's Medium-Glucose Free) was prepared by first warming the medium in a water bath, in part to reduce the solubility of oxygen in the liquid medium. 100% nitrogen was bubbled for 30 minutes through the medium using a 0.5 μm diffusing stone to remove dissolved oxygen. HEPES buffer was added to a final concentration of 1 mM. Medium was added directly to the neurons at the end of the sparge. A small sample of the medium was aliquoted for confirmation of oxygen levels using a dip-type oxygen sensor. Oxygen levels were typically reduced to 0.9% to about 5.0% oxygen.

A hypoxia chamber was prepared by placing the chamber in an incubator at 37° C. for at least 4 hours (overnight preferred) prior to gassing. Medium in the culture vessels was removed and replaced with de-gassed medium, and the culture vessels were placed in the hypoxia chamber. The hypoxia chamber was then flushed with 95% N₂/5% CO₂ gas through the system at a rate of 20-25 Lpm for at least 5 minutes. The system was incubated in the incubator at 37° C. for 4 hours, with degassing of the chamber once more after 1 hour.

At the conclusion of the insult procedure, OGD medium was aspirated and warm medium was added to the neurons. 24-28 hours later, AMDACs and neurons were plated at equal numbers at 100,000 cells each per well of a 6-well plate suspended in Neuronal Medium were added to the neurons and co-cultured for 6 days.

Photomicrographs were taken of random fields in a 6-well plate for each condition. Cells having a typical neuron morphology were identified, and neurite lengths were recorded. The average length of the neurites positively correlated to neuronal health, and were longer in co-cultures of neurons and AMDACs, indicating that the AMDACs were protecting the cells from the insult.

### Reactive Oxygen Species Assay

AMDACs were determined to express superoxide dismutase, catalase, and heme oxygenase gene during hypoxia. The ability of AMDACs to scavenge reactive oxygen species, and to protect cells from such species, was determined in an assay using hydrogen peroxide as a reactive oxygen species generator.

Assay Description: Target cells (Astrocytes, ScienCell Research Laboratories) were seeded in 96-well black well plates pre-coated with poly-L-lysine at 6000/cm². The astrocytes are allowed to attach overnight in growth medium at 37° C. with 5% carbon dioxide. The following day, the culture media was removed and the cells were incubated with cell permeable dye DCFH-DA (Dichlorofluorescin diacetate), which is a fluorogenic probe. Excess dye was removed by washing with Dulbecco's Phosphate Buffered Saline or Hank's Buffered Salt Solution. The cells were then insulted with reactive oxygen species by addition of 1000 μM hydrogen peroxide for 30-60 minutes. The hydrogen peroxide-containing medium was then removed, and replaced with serum-free, glucose-free growth medium. AMDACs (either cells designated as Lot 1 or Lot 2), or BM-MSCs, were added at 6000/cm², and the cells were cultured for another 24 hours. The cells were then read in a standard fluorescence plate reader at 480Ex and 530Em. The reactive oxygen species content of the medium was directly proportional to the levels of DCFH-DA in the cell cytosol. The reactive oxygen species content was measured by comparison to pre-determined DCF standard curve. All experiments were done with N=24.

For the assay, 1×DCFH-DA was prepared immediately prior to use by diluting a 20×DCFH-DA stock solution to 1× in cell culture media without fetal bovine serum, and stirring to homogeneity. Hydrogen Peroxide (H₂0₂) dilutions were prepared in DMEM or DPBS as necessary. A standard curve was prepared as a 1:10 dilution series in concentration range 0 μM to 10 μM by diluting 1 mM DCF standard in cell culture media, transferring 100 μl of DCF standard to a 96 well plate suitable for fluorescent measurement, and adding 100 μl of cell lyses buffer. Fluorescence was read at 480Ex and 530Em.

Results: Both lots of AMDACs used significantly reduced the concentration of reactive oxygen species in the astrocyte co-cultures. In contrast, BM-MSCs failed to significantly reduce reactive oxygen species in the astrocyte co-cultures.

### EQUIVALENTS

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed is:

1. A method of treating an individual having schizophrenia or schizophreniform disorder, comprising administering to the individual a therapeutically effective amount of amnion derived adherent cells (AMDACs), or culture medium conditioned by AMDACs, wherein the therapeutically effective amount is an amount sufficient to cause a detectable improvement in one or more symptoms of said schizophrenia or schizophreniform disorder manifested in the individual, and wherein said AMDACs are CD49f⁺ as determinable by flow cytometry, and OCT-4⁻ as determinable by PCR.

2. The method of claim 1, wherein said individual has been diagnosed as having schizophrenia or schizophreniform disorder according to criteria listed in the Diagnostic and Statistical Manual of Mental Disorders (DSM).

3. The method of claim 1, wherein said one or more symptoms comprise one or more Schneiderian First Rank symptoms.

4. The method of claim 3, wherein said one or more symptoms comprise one or more of hearing thoughts audibly; hearing voices arguing; hearing voices commenting on one's actions; delusions of being controlled by others; delusions that the individual's thoughts have been withdrawn or taken by others; delusions that thoughts have been inserted or caused by others; delusions that others can read the individual's thoughts; or delusional perception by said individual.

5. The method of claim 1, wherein said one or more symptoms comprise one or more of disordered thoughts and/or speech; tactile hallucinations; auditory hallucinations; visual hallucinations; olfactory hallucinations; gustatory hallucinations); flat or blunted affect; flat or blunted emotion; poverty of speech; inability to experience pleasure; lack of desire to form relationships; or lack of motivation.

6. The method of claim 1, wherein said cell is HLA-G⁻, as determinable by RT-PCR.

7. The method of claim 1, wherein said cell is additionally CD49f⁺, as determinable by flow cytometry.

8. The method of claim 7, wherein said AMDACs are OCT-4⁻, HLA-G⁻ and CD49f⁺.

9. The method of claim 1, wherein said AMDACs are CD90⁺, CD105⁺, or CD117⁻ as determinable by flow cytometry.

10. The method of claim 9, wherein said AMDACs are CD90⁺, CD105⁺, and CD11 T as determinable by flow cytometry.

11. The method of claim 10, wherein said AMDACs are OCT-4⁻ and HLA-G⁻, as determined by RT-PCR, and CD49f⁺, CD90⁺, CD105⁺, and CD117⁻ as determinable by flow cytometry.

12. The method of claim 1, wherein said AMDACs are VEGFR1/Flt-1⁺ (vascular endothelial growth factor receptor 1) and VEGFR2/KDR⁺ (vascular endothelial growth factor receptor 2), as determinable by immunolocalization.

13. The method of claim 1, wherein said AMDACs are one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻ (angiotensin-I-converting enzyme, ACE), CD146⁻ (melanoma cell adhesion molecule), or CXCR4⁻ (chemokine (C-X-C motif) receptor 4) as determinable by immunolocalization.

14. The method of claim 1, wherein said AMDACs are CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, and CXCR4⁻ as determinable by immunolocalization.

15. The method of claim 1, wherein said cell is VE-cadherin⁻ as determinable by immunolocalization.

16. The method of claim 1, wherein said AMDACs are additionally positive for CD105⁺ and CD200⁺ as determinable by immunolocalization.

17. The method of claim 1, wherein said AMDACs do not express CD34 as determinable by immunolocalization after exposure to 50 ng/mL VEGF for 7 days.

18. The method of claim 1, wherein said AMDACs are adherent to tissue culture plastic; wherein said cell is OCT-4⁻, as determined by RT-PCR, and CD49f⁺, HLA-G⁻, CD90⁺, CD105⁺, and CD117⁻, as determined by immunolocalization; and wherein said AMDACs:
(a) express one or more of CD9, CD10, CD44, CD54, CD98, CD200, Tie-2, TEM-7, VEGFR1/Flt-1, or VEGFR2/KDR (CD309), as determinable by immunolocalization;
(b) lack expression of CD31, CD34, CD38, CD45, CD133, CD143, CD144, CD146, CD271, CXCR4, HLA-G, or VE-cadherin, as determinable by immunolocalization, or lack expression of SOX2, as determinable by RT-PCR;
(c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR;
(d) express one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type JIB precursor, Wnt-9 protein, glial fibrillary acidic protein, astrocyte, myosin-binding protein C, or myosin heavy chain, nonmuscle type A;
(e) secrete VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the AMDACs are cultured;
(f) express micro RNAs miR-17-3p, miR-18a, miR-18b, miR-19b, miR-92, or miR-296 at a higher level than an equivalent number of bone marrow-derived mesenchymal stem cells;
(g) express micro RNAs miR-20a, miR-20b, miR-221, miR-222, miR-15b, or miR-16 at a lower level than an equivalent number of bone marrow-derived mesenchymal stem cells;
(h) express miRNAs miR-17-3p, miR-18a, miR-18b, miR-19b, miR-92, miR-20a, miR-20b, miR-296, miR-221, miR-222, miR-15b, or miR-16; or
(i) express increased levels of CD202b, IL-8 or VEGF when cultured in less than about 5% O₂, compared to expression of CD202b, IL-8 or VEGF under 21% O₂.

19. The method of claim 18, wherein said AMDACs are OCT-4⁻, as determined by RT-PCR, and CD49f⁺, HLA-G⁻, CD90⁺, CD105⁺, and CD117⁻, as determinable by immunolocalization, and wherein said AMDACs:
(a) express CD9, CD10, CD44, CD54, CD98, CD200, Tie-2, TEM-7, VEGFR1/Flt-1, and/or VEGFR2/KDR (CD309), as determinable by immunolocalization;
(b) lack expression of CD31, CD34, CD38, CD45, CD133, CD143, CD144, CD146, CD271, CXCR4, HLA-G, and/or VE-cadherin, as determinable by immunolocalization, and/or lack expression of SOX2, as determinable by RT-PCR;
(c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, and/or VEGFR2/KDR;
(d) express one or more of CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type JIB precursor, Wnt-9 protein, glial fibrillary acidic protein, astrocyte, myosin-binding protein C, and/or myosin heavy chain, nonmuscle type A;
(e) secrete one or more of VEGF, HGF, IL-8, MCP-3, FGF2, Follistatin, G-CSF, EGF, ENA-78, GRO, IL-6, MCP-1, PDGF-BB, TIMP-2, uPAR, and Galectin-1 into culture medium in which the AMDACs are cultured;
(f) express micro RNAs miR-17-3p, miR-18a, miR-18b, miR-19b, miR-92, and/or miR-296 at a higher level than an equivalent number of bone marrow-derived mesenchymal stem cells;
(g) express micro RNAs miR-20a, miR-20b, miR-221, miR-222, miR-15b, and/or miR-16 at a lower level than an equivalent number of bone marrow-derived mesenchymal stem cells;
(h) express miRNAs miR-17-3p, miR-18a, miR-18b, miR-19b, miR-92, miR-20a, miR-20b, miR-296, miR-221, miR-222, miR-15b, and/or miR-16; and
(i) express increased levels of CD202b, IL-8 and/or VEGF when cultured in less than about 5% O₂, compared to expression of CD202b, IL-8 and/or VEGF under 21% O₂.

20. A therapeutically effective amount of amnion-derived adherent cells, or culture medium conditioned by amnion derived adherent cells, for use in treating an individual having schizophrenia or schizophreniform disorder, wherein the therapeutically effective amount is an amount sufficient to cause a detectable improvement in one or more symptoms of said schizophrenia or schizophreniform disorder manifested by said individual, and wherein said AMDACs are CD49f⁺ as determinable by flow cytometry, and OCT-4⁻ as determinable by PCR.

21. The amnion derived adherent cells or conditioned culture medium of claim 20, wherein said individual has been diagnosed as having schizophrenia or schizophreniform disorder according to criteria listed in the Diagnostic and Statistical Manual of Mental Disorders (DSM).

22. The amnion derived adherent cells or conditioned culture medium of claim 20, wherein said one or more symptoms comprise one or more Schneiderian First Rank symptoms.

23. The amnion derived adherent cells or conditioned culture medium of claim 20, wherein said one or more symptoms comprise one or more of hearing thoughts audibly; hearing voices arguing; hearing voices commenting on one's actions; delusions of being controlled by others; delusions that the individual's thoughts have been withdrawn or taken by others; delusions that thoughts have been inserted or caused by others; delusions that others can read the individual's thoughts; or delusional perception by said individual.

24. The amnion derived adherent cells or conditioned culture medium claim 20, wherein said one or more symptoms comprise one or more of disordered thoughts and/or speech; tactile hallucinations; auditory hallucinations; visual hallucinations; olfactory hallucinations; gustatory hallucinations); flat or blunted affect; flat or blunted emotion; poverty of speech; inability to experience pleasure; lack of desire to form relationships; or lack of motivation.

25. The amnion derived adherent cells or conditioned culture medium of claim 20, wherein said cell is HLA-G⁻, as determinable by RT-PCR.

26. The amnion derived adherent cells or conditioned culture medium of claim 20, wherein said cell is additionally CD49f⁺, as determinable by flow cytometry.

27. The amnion derived adherent cells or conditioned culture medium of claim 26, wherein said AMDACs are OCT-4⁻, HLA-G⁻ and CD49f⁺.

28. The amnion derived adherent cells or conditioned culture medium of claim 20, wherein said AMDACs are CD90⁺, CD105⁺, or CD117⁻ as determinable by flow cytometry.

29. The amnion derived adherent cells or conditioned culture medium of claim 28, wherein said AMDACs are CD90⁺, CD105⁺, and CD117⁻ as determinable by flow cytometry.

30. The amnion derived adherent cells or conditioned culture medium of claim 29, wherein said AMDACs are OCT-4⁻ and HLA-G⁻, as determined by RT-PCR, and CD49f⁺, CD90⁺, CD105⁺, and CD117⁻ as determinable by flow cytometry.

31. The amnion derived adherent cells or conditioned culture medium of claim 20, wherein said AMDACs are VEGFR1/Flt-1⁺ (vascular endothelial growth factor receptor 1) and VEGFR2/KDR⁺ (vascular endothelial growth factor receptor 2), as determinable by immunolocalization.

32. The amnion derived adherent cells or conditioned culture medium of claim 20, wherein said AMDACs are one or more of CD9 CD10⁺, CD44⁺, CD54⁺, CD98 Tie-2⁺ (angiopoietin receptor), TEM-7⁺ (tumor endothelial marker 7), CD31⁻, CD34⁻, CD45⁻, CD133⁻, CD143⁻ (angiotensin-I-converting enzyme, ACE), CD146⁻ (melanoma cell adhesion molecule), or CXCR4⁻ (chemokine (C-X-C motif) receptor 4) as determinable by immunolocalization.

33. The amnion derived adherent cells or conditioned culture medium of claim 20, wherein said AMDACs are CD9⁺ CD10⁺, CD44⁺, CD54⁺, CD98 Tie-2⁺ (angiopoietin receptor), TEM-7⁺ (tumor endothelial marker 7), CD31⁻, CD34⁻, CD45⁻, CD133⁻, CD143⁻, CD146⁻, and CXCR4⁻ as determinable by immunolocalization.

34. The amnion derived adherent cells or conditioned culture medium of claim 20, wherein said cells are VE-cadherin⁻ as determinable by immunolocalization.

35. The amnion derived adherent cells or conditioned culture medium of claim 20, wherein said AMDACs are additionally positive for CD105 and CD200⁺ as determinable by immunolocalization.

36. The amnion derived adherent cells or conditioned culture medium of claim 20, wherein said AMDACs do not express CD34 as determinable by immunolocalization after exposure to 50 ng/mL VEGF for 7 days.

37. The amnion derived adherent cells or conditioned culture medium of claim 20, wherein said AMDACs are adherent to tissue culture plastic; wherein said cell is OCT-4⁻, as determined by RT-PCR, and CD49f⁺, HLA-G⁻, CD90⁺, CD105⁺ and CD117⁻, as determined by immunolocalization; and wherein said AMDACs:
(a) express one or more of CD9, CD10, CD44, CD54, CD98, CD200, Tie-2, TEM-7, VEGFR1/Flt-1, or VEGFR2/KDR (CD309), as determinable by immunolocalization;
(b) lack expression of CD31, CD34, CD38, CD45, CD133, CD143, CD144, CD146, CD271, CXCR4, HLA-G, or VE-cadherin, as determinable by immunolocalization, or lack expression of SOX2, as determinable by RT-PCR;
(c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR;
(d) express one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type JIB precursor, Wnt-9 protein, glial fibrillary acidic protein, astrocyte, myosin-binding protein C, or myosin heavy chain, nonmuscle type A;
(e) secrete VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the AMDACs are cultured;
(f) express micro RNAs miR-17-3p, miR-18a, miR-18b, miR-19b, miR-92, or miR-296 at a higher level than an equivalent number of bone marrow-derived mesenchymal stem cells;
(g) express micro RNAs miR-20a, miR-20b, miR-221, miR-222, miR-15b, or miR-16 at a lower level than an equivalent number of bone marrow-derived mesenchymal stem cells;
(h) express miRNAs miR-17-3p, miR-18a, miR-18b, miR-19b, miR-92, miR-20a, miR-20b, miR-296, miR-221, miR-222, miR-15b, or miR-16; or
(i) express increased levels of CD202b, IL-8 or VEGF when cultured in less than about 5% O₂, compared to expression of CD202b, IL-8 or VEGF under 21% O₂.

38. The amnion derived adherent cells or conditioned culture medium of claim 37, wherein said AMDACs are OCT-4⁻, as determined by RT-PCR, and CD49f⁺, HLA-G⁻, CD90⁺, CD105⁺, and CD117⁻, as determinable by immunolocalization, and wherein said AMDACs:
(a) express CD9, CD10, CD44, CD54, CD98, CD200, Tie-2, TEM-7, VEGFR1/Flt-1, and/or VEGFR2/KDR (CD309), as determinable by immunolocalization;
(b) lack expression of CD31, CD34, CD38, CD45, CD133, CD143, CD144, CD146, CD271, CXCR4, HLA-G, and/or VE-cadherin, as determinable by immunolocalization, and/or lack expression of SOX2, as determinable by RT-PCR;
(c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, and/or VEGFR2/KDR;
(d) express one or more of CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A;
(e) secrete one or more of VEGF, HGF, IL-8, MCP-3, FGF2, Follistatin, G-CSF, EGF, ENA-78, GRO, IL-6, MCP-1, PDGF-BB, TIMP-2, uPAR, and Galectin-1 into culture medium in which the AMDACs are cultured;
(f) express micro RNAs miR-17-3p, miR-18a, miR-18b, miR-19b, miR-92, and/or miR-296 at a higher level than an equivalent number of bone marrow-derived mesenchymal stem cells;
(g) express micro RNAs miR-20a, miR-20b, miR-221, miR-222, miR-15b, and/or miR-16 at a lower level than an equivalent number of bone marrow-derived mesenchymal stem cells;
(h) express miRNAs miR-17-3p, miR-18a, miR-18b, miR-19b, miR-92, miR-20a, miR-20b, miR-296, miR-221, miR-222, miR-15b, and/or miR-16; and
(i) express increased levels of CD202b, IL-8 and/or VEGF when cultured in less than about 5% O₂, compared to expression of CD202b, IL-8 and/or VEGF under 21% O₂.
